(19) United States
(12) Patent Application Publication
(10) Pub. No.: US 2013/0225416 A1
(43) Pub. Date: Aug. 29, 2013

(54) ELECTRONIC SEQUENCING

(71) Applicants: Gabriela ALTMANN, Berlin (DE); Oliver HAYDEN, Herzogenaurach (DE); Oliver LADE, Berlin (DE); Christian P. SCHULTZ, Beverly, MA (US); Gerald WARNAT, Ahlerstedt (DE)

(72) Inventors: Gabriela ALTMANN, Berlin (DE); Oliver HAYDEN, Herzogenaurach (DE); Oliver LADE, Berlin (DE); Christian P. SCHULTZ, Beverly, MA (US); Gerald WARNAT, Ahlerstedt (DE)

(21) Appl. No.: 13/688,862

(22) Filed: Nov. 29, 2012

Related U.S. Application Data
(60) Provisional application No. 61/564,403, filed on Nov. 29, 2011.

(51) Int. Cl. C12Q 1/68

(52) U.S. Cl. CPC C12Q 1/6874
USPC 506/2; 506/16

(57) ABSTRACT

A method for sequencing nucleic acid molecules a) amplifies nucleic acid molecules and b) sequences the amplified nucleic acid molecules. Steps (a) and (b) are carried out on an array of field effect transistor (FET) sensor elements, comprising an array of nanowires. Within the array, each FET may include at least one nanowire, or one each FETs can lie between two nanowires, or one nanowire of a nanowire pair is a nanowire FET, with nucleic acids bound to a nanowire surface. A chip includes one or more arrays of field effect transistor sensor elements. A kit for sequencing nucleic acid molecules, may include one or more amplifying reagents or sequencing reagents.

### CROSS REFERENCE TO RELATED APPLICATIONS

This application is based on and hereby claims priority to U.S. Provisional Application No. 61/564,403 filed on Nov. 29, 2011, the contents of which are hereby incorporated by reference.

### BACKGROUND

To date the technique of nucleic acid sequencing has had far-reaching effects on research in the life sciences, biotechnology, and in medicine. The aim of reducing the costs and expenditure of time for DNA sequencing has led to a series of new sequencing methods. Although as a result the costs for DNA sequencing have already fallen considerably, the desire still remains to reduce the costs for DNA sequencing in the future, too, and furthermore to make available faster and portable devices for DNA sequencing in order to realize the aim of the $1000 genome.

This challenge has led to the development of next generation sequencing (NGS) technologies. The cost-effective production of large amounts of sequence data is the primary advantage over conventional methods.

The method of bridge amplification is known from US 2011045541 A1. This involves hybridizing nucleic acid molecules to corresponding immobilized primers with subsequent elongation of the immobilized primer, wherein the nucleic acid molecule serves as a template. After the elongated primer has been separated from the nucleic acid template, the elongated primer can once again hybridize to another immobilized primer to form a bridge and this other primer can be elongated, wherein this time the elongated primer serves as a template. The two extended primers, which form a double strand to form a bridge, can then be separated from one another and are available as nucleic acid templates in the next round of amplification. The method is repeated in order to make available colonies of immobilized nucleic acid molecules, including both forward and reverse strands. However, it is only possible to sequence the sequence of only one strand, either forward or reverse strand of said colony, and not both the forward strand and the reverse strand. Moreover, this solid phase amplification gives rise to a stochastic distribution of the immobilized primers on the surface, thus also resulting in stochastic colony formation made of forward and reverse strands. The basis of this is the stochastic distribution of the primers. The sequencing is performed with fluorescence-marked nucleotides, such that label-free detection is not possible.

Hitherto, imaging methods, electromagnetic intermediate products (either X-rays or light) and particular nucleotides or other reagents have been the limiting factors for the methods for nucleic acid sequencing. In order to overcome these obstacles, non-optical sequencing methods based on newly developed integrated circuits are striven for in practice. It is known that ion-sensitive field effect transistors (ISFET) are suitable electronic sensor elements for identifying the hydrogen ions (H+) liberated during the sequencing by synthesis by the polymerase from the OH group at the 3′ end of the DNA molecule during the formation of the phosphodiester bond. The liberated H+ cause a charge cloud and lead to a change in the pH value and they induce a change in the surface potential at the surface of the field effect transistor (FET). It is thus possible to measure the change in the surface potential using the FET. Generally, FETs exhibit a change in conductivity in reaction to changes in the electric field or potential at the surface. In a standard FET, a semiconductor, such as e.g. p-type silicon (p-Si), is connected to metal electrodes, source and drain, through which current is introduced and collected. The conductivity of the semiconductor between source and drain is switched on and off by a third, gate electrode, which is capacitively coupled by a thin dielectric layer. The dependence of the conductivity on the gate voltage turns FETs into electrical sensor elements, since the electric field resulting from the binding of a charged species to the gate dielectric is analogous to the effects resulting from applying a voltage with a gate electrode. This concept for measurement using FETs was applied to chemically sensitive field effect transistors (ChemFET) a few decades ago. However, the two-dimensional, planar devices have only a limited sensitivity, which has prevented ChemFETs from being able hitherto to manifest a great effect as chemical or biological sensor elements.

WO 2010008480 A2 discloses using FET arrays for analyte measurements. The FET arrays are used in methods for DNA sequencing which are based on the monitoring of changes in concentration of inorganic pyrophosphate (PPa), hydrogen ions (H+) and nucleoside triphosphate (NTP). In this case, nucleic acid templates are arranged in reaction chambers. The reaction chambers are in contact with or are capacitively coupled to at least one ChemFET within an array of ChemFETs. The nucleic acid templates are hybridized to sequencing primers and bound to a polymerase. A new nucleic acid strand is synthesized by one or more known nucleoside triphosphates being incorporated successively at the 3′ end of the sequencing primer. The incorporation is detected by measuring the change in an electrical parameter at a ChemFET, no appreciable change in pH taking place in the reaction chamber. In this case, the nucleic acid templates are bound to individual beads, wherein a bead is in each case situated in a reaction chamber. For carrying out the nucleic acid sequencing, it is necessary to amplify the nucleic acid in a preceding step. The nucleic acids to be sequenced are bound to beads and clonally amplified in an emulsion PCR. Afterward, the beads are positioned in wells above an individual FET for the sequencing.

However, this method lengthens the times for the sequential synthesis of the new nucleic acid strands on account of the necessary diffusion time and the necessary washing steps owing to the laminar conditions and the dimensioning of the wells. The use of a bulky structure such as that of a bead results in an unfavorable surface area-to-volume ratio, which has the effect that the sensitivity of the ChemFET is reduced. Furthermore, the FETs are two-dimensional or planar FETs which have a lower sensitivity than a one-dimensional FET, such as e.g. a nanowire FET. WO 2010008480 A2 does not describe that it is possible to carry out both the upstream clonal nucleic acid amplification and the nucleic acid sequencing on a single FET platform.

### SUMMARY

One potential problem is providing a label-free sequencing method and enabling both an upstream clonal nucleic acid amplification and a nucleic acid sequencing to be carried out in an integrated fashion on a single FET platform having an improved sensitivity.

Another potential problem is providing an array of field effect transistor (FET) sensor elements comprising an array of nanowires, a chip, a device and a kit, and of enabling the use thereof for carrying out the label-free sequencing method.

The inventors propose a method for sequencing nucleic acid molecules, comprising the following steps:
a) amplifying one or more nucleic acid molecules;
b) sequencing one or more of the amplified nucleic acid molecules,

wherein steps (a) and (b) are carried out on an array of field effect transistor (FET) sensor elements, comprising an array of nanowires.

For carrying out the method, an array of field effect transistor (FET) sensor elements, comprising an array of nanowires, is made available, wherein at least one FET comprises at least one nanowire, wherein one or more FETs lie between in each case two nanowires, or wherein at least one nanowire of a nanowire pair is a nanowire FET, and wherein one or more nucleic acids are bound to at least one nanowire surface.

A further aspect is a chip, comprising one or more arrays of field effect transistor (FET) sensor elements.

Furthermore, a device comprising one or more chips is provided.

Another aspect relates to a kit for sequencing nucleic acid molecules, which provides one or more arrays of field effect transistor (FET) sensor elements, one or more chips, or a device and one or more amplifying reagents or sequencing reagents.

The array of field effect transistor (FET) sensor elements, the chip, the device or the kit can thus be used for amplifying and/or sequencing nucleic acid molecules and/or for analyzing nucleic acids.

The advantages achieved relate to the fact that a sequencing method is made possible which allows the integration of a directed clonal bridge amplification and a nucleic acid sequencing on a single FET platform, in particular on an array of field effect transistor (FET) sensor elements, comprising an array of nanowires. This enables a directed clonal bridge amplification and an electrical real-time read-out of the bridge formation and of the nucleic acid sequencing by a field effect of the FET.

A further advantage of the method is that, both during the bridge amplification and during the bridge sequencing, the nucleoside triphosphates need not be marked, e.g. by radioactive, dye- or fluorescence-marked or other probes, in order to detect the incorporation thereof into a nucleic acid molecule. The method therefore enables marking-free/label-free detection since H+ are liberated during the synthesis of the nucleic acid strands in the course of the bridge amplification and the bridge sequencing by a polymerase. Said H+ form a charge cloud, which leads to a change in the pH value and induces a change in the surface potential at the surface of the FET, which can be measured by the FET.

The use of virtually one-dimensional nanowires results in a more favorable surface area-to-volume ratio compared with a two-dimensional system, which increases the sensitivity of the FET platform. The high field sensitivity enables a quantitative PCR or a nested PCR or a PCR followed by sequencing on a single FET platform.

The higher sensitivity of a FET based on nanowires results in a read length of preferably >1 kb. In contrast to two-dimensional FETs, one-dimensional nanowires avoid the reduction of conductivity that is caused by lateral parallel connection of currents. The cause of the increase in sensitivity is the maximum surface area-to-volume ratio of the nanowires. The nanowire sensitivity is dependent on the device diameter and this, for a device of specific length and constant space charge density, scales with the reciprocal of the nanowire radius. The geometry of the nanowires limits the current flow to a much thinner region than in the mass or area, and the molecules adsorbed on the nanowire surface produce more significant effects.

The use of a nanowire FET makes it possible to permit the formation of bridges during the nucleic acid amplification and the nucleic acid sequencing in a positionally directed manner from one nanowire to another nanowire by forming an electric field. This is not possible in the related art methods involving stochastic colony formation, since the bridges form stochastically as a result of random distribution of the immobilized primers.

In contrast to methods which use reaction chambers with nucleic acid templates coupled to beads, there is a lower probability of dephasing since no NTP depletion zone can form in the region between bead and well and by virtue of the fact that there is a higher ion intensity during synthesis. Dephasing occurs during the step-by-step addition of NTPs for elongation of the primers. In this case, it can happen that the growing primers depart from synchronicity in one of the cycles of the NTP addition. This can result in incomplete nucleotide strands or nucleotide strands in which a plurality of nucleotides are incorporated in one cycle.

The spatial fixing of the nucleic acid templates during sequencing keeps the concentrated H+ charge cloud that arises in FET proximity and thus enables a further increase in sensitivity.

As used herein, the definite article “the” or the indefinite articles “a”, “an” or the like refer to “one” or “a plurality”, i.e. they mean “one”, “at least one” or “one or more”. By way of example, the expression “the nucleic acid primer” can refer to an individual nucleic acid primer or to a multiplicity of nucleic acid primers, irrespective of whether it is specifically pointed out in an embodiment that one or more nucleic acid primers are used.

In all embodiments, the words “comprising”, “including”, “encompassing” or “containing” can also have the meaning of “consisting of”. That is to say that in these embodiments the presence of an additional component can be precluded.

The term “nucleic acids”, as used herein, refers to any form of DNA, cDNA, RNA, PNA (peptide nucleic acid), LNA (locked nucleic acid), GNA (glycol nucleic acid), TNA (threose nucleic acid), synthetic nucleic acids or derivatives thereof.

The German application text uses the German term “Templat” and the English term “template” interchangeably. Both terms denote a matrix, e.g. a nucleic acid strand, which is used for duplicating nucleotide sequences.

The expression “adapter”, as used herein, refers to a terminal or internal sequence of a nucleic acid molecule, usually of one single-stranded nucleic acid molecule, which is complementary to a sequence of another nucleic acid molecule, usually of a primer, and can therefore hybridize thereto.

The term “read”, as used herein, denotes a nucleic acid sequence that is read.

The term “marking” is used herein synonymously with “label” and refers to any chemical group which can be identified with the aid of a suitable optical detection system. The marking does not occur in naturally occurring nucleotides. Such markings are radioactive or non-radioactive markings, such as e.g. fluorescent dyes, fluorophores.

The term FET platform, as used herein, means any hardware on the surface of which at least one field effect transistor is situated. In particular, the term FET platform encompasses an array of field effect transistor (FET) sensor elements, which can comprise e.g. an array of nanowires.

As used herein, the term “bound”, “bind” or “binding” means a binding which can be selected from a covalent bond, an electrostatic interaction or a weak interaction, such as e.g. a van der Waals interaction, a dipole interaction or a hydrogen bridge bond or a mixture of these three types of binding, such as e.g. in the binding of biotin to streptavidin.

As used herein, the term “sensor element” means an individual field effect transistor (FET). The term “array of field effect transistor (FET) sensor elements”, as used herein, comprises an arrangement of a multiplicity of sensor elements or FETs. The term “sensor” refers to the surface of a chip, on which surface one or more arrays of field effect transistor (FET) sensor elements are situated.

The expressions “first nanowire”, “second nanowire”, “third nanowire”, “fourth nanowire” or “first nanowire surface”, “second nanowire surface”, “third nanowire surface”, “fourth nanowire surface” refer to a relative, spatial sequence or order of the nanowires, with the nanowire surfaces thereof relative to one another, within a nanowire array. However, the expression “first nanowire” does not necessarily refer to the very first nanowire of a nanowire array and the expression “second nanowire” does not necessarily refer to the nanowire that follows the very first nanowire of a nanowire array. By way of example, the “first nanowire” can have further nanowires adjacent on the left and the “fourth nanowire” can have further nanowires adjacent on the right. Since the designated sequence is relative, a further nanowire can also lie between a first nanowire and a second nanowire. Since the order is also relative, for example a first nanowire can be designated as a fourth nanowire and a fourth nanowire can be designated as a first nanowire.

Otherwise, the use of the numerical terms “first”, “second”, “third”, etc. serves to distinguish different elements from one another; e.g. the expression “first” and “second” nucleic acid primer refers to mutually different nucleic acid primers. The terms such as “first”, “second”, etc. does not necessarily mean a spatial sequence or order.

As used herein, the expression “adjacent” should be understood to mean that if one nanowire or one nanowire surface lies “adjacent” to another nanowire or another nanowire surface, both the terms “directly” and “indirectly” adjacent are included.

The proposed method for sequencing nucleic acid molecules, comprises the following steps:
a) amplifying one or more nucleic acid molecules;
b) sequencing one or more of the amplified nucleic acid molecules,

wherein steps (a) and (b) are carried out on an array of field effect transistor (FET) sensor elements, comprising an array of nanowires.

The array of nanowires is part of an array of field effect transistor (FET) sensor elements. The nanowires are part of a FET; e.g. they can form the gate of a FET or the nanowires are the voltage source for driving a gate of a FET.

Preferably, the method for sequencing nucleic acid molecules is a marking-free/label-free method. In particular, step (b) of the sequencing of one or more of the amplified nucleic acid molecules is marking-free. This makes it possible to measure inherent properties of the molecules, e.g. mass or dielectric properties. Moreover, a chemical modification of the relevant molecules with markers is no longer necessary.

Preferably, all the steps of the method as described below are performed in a microfluidic environment. This has the advantage that the use of reagents can be minimized, and that the sequential work steps are processed rapidly.

The inventors propose for steps (a) and (b) of the method for sequencing a nucleic acid molecule to be carried out on an array of nanowires, in particular on the surface of the nanowires. The use of virtually one-dimensional nanowires increases the sensitivity of the system compared with a two-dimensional system.

In accordance with a first embodiment, the nanowires of the array for carrying out the method are semiconductor or metal nanowires. Semiconductor nanowires have the potential also to serve as highly sensitive and selective sensor elements for the marking-free detection of low concentrations. The nucleic acid amplification in step (a) or the nucleic acid sequencing in step (b) can be carried out on metal nanowires or on semiconductor nanowires. A FET can comprise a semiconductor nanowire. An array of semiconductor nanowires corresponds to an array of field effect transistor (FET) sensor elements. If the nucleic acid amplification in step (a) or the nucleic acid sequencing in step (b) is performed on the surfaces of metal nanowires, at least one semiconductor nanowire is situated between in each case two such metal nanowires. The metal nanowires are not part of a FET in this case, rather for the electronic read-out semiconductor nanowire FETs lie between the metal nanowires.

A nanowire array preferably comprises at least four adjacent, parallel nanowires, wherein the first nanowire can contain further nanowires adjacent to the left of it and the fourth nanowire can contain further nanowires adjacent to the right of it. In preferred embodiments, the array of nanowires comprises at least four or at least six adjacent, parallel nanowires. Hereinafter, four adjacent, parallel nanowires are designated, for simplification, as first, second, third and fourth nanowires having a first, second, third or fourth nanowire surface, wherein this designation should not be regarded as restrictive, i.e. a first nanowire does not necessarily represent the nanowire number one of a nanowire array. It is understood that the order of the nanowires can also be read backward.

In one embodiment, before step (a) of the method is carried out, an array of nanowires is made available, wherein the nanowires are coated with a polymer, preferably polytetrafluoroethylene (PTFE). The coating with a polymer prevents the binding of molecules, in particular nucleic acid molecules, to the nanowire surface.

The polymer layer preferably has a thickness of less than 100 nm, preferably of less than 50 nm, particularly preferably of less than 40 nm. In one particular embodiment, the polymer coating has a thickness of 30 nm.

Preferably, the polymer is ablatable by Joule heating. In this case, “ablatable” should be understood to mean a layer which is removed or destroyed by the effect of the Joule heating, such that the nanowire is preferably uncoated again.

During Joule heating, as described herein, an electric current is conducted through a nanowire, wherein the polymer coating is ablated. For an individual nanowire, Joule heating takes place at less than 10 V/μm nanowire length, less than 5 V/μm nanowire length, less than 3 V/μm nanowire length, most preferably at 2.5 V/μm nanowire length.

In this case, Joule heating takes place to less than 100° C., less than 200° C., less than 300° C., less than 400° C., less than 500° C. or less than 600° C. Most preferably, Joule heating takes place to 325° C.

In particular embodiments, the lateral resolution of the polymer layer around a nanowire is less than 1000 nm, less than 800 nm, less than 500 nm, less than 400 nm or less than 300 nm. Most preferably, the lateral resolution of the polymer layer around a nanowire is 200 nm.

Each of the nanowires of a nanowire array is selectively ablatable. That means that in a nanowire array the surface can be selectively functionalized by only a specific nanowire being ablated, while the surrounding surfaces and thus also the other nanowire surfaces retain their polymer coating. The binding of a nucleic acid can take place on the ablated nanowire surface, but not on the surfaces of other nanowires that bear a polymer coating. This enables the addressability of each individual nanowire by ablation by Joule heating. Joule heating is used in order to selectively remove a polymer coating on a specific nanowire surface. The selective ablatability enables successively in the process the selective addressability of the different nanowires with in each case different nucleic acid primers. By way of example, the activation of an array of nanowires by an ablation of the polymer layer also allows a sequential and patient-number-dependent addressability for e.g. specific sequence searches or an individualized sequencing depth.

The method comprises step (a) a directed clonal bridge amplification. The term “directed”, as used herein, means that the use of a FET makes it possible to apply an electric field and to align the nucleic acid strands that carry a negative charge on an oxygen atom of the phosphate group of the nucleic acid strand toward the positive pole of the electric field and to hybridize, fix and/or amplify the single-strand nucleic acid template or templates clonally in a targeted manner at precisely predefined nanowire surfaces.

In contrast to stochastic formation of a bridge, in the case of directed bridge formation a single-stranded nucleic acid strand bound to the surface of one nanowire and a nucleic acid primer bound to the surface of another, adjacent nanowire can be aligned by an electric field such that the hybridization of the single-stranded nucleic acid strand to the nucleic acid primer is controlled with the formation of a bridge.

In embodiments, before the step of binding a nucleic acid primer to a nanowire surface, the clonal bridge amplification comprises the following step: ablating the polymer coating of the nanowire surface by Joule heating.

In embodiments, the directed clonal bridge amplification comprises the following step: selectively binding a first nucleic acid primer to one nanowire surface, for example a second nanowire surface.

A further embodiment comprises selectively binding a second nucleic acid primer to another, adjacent nanowire surface, for example a third nanowire.

Preferably, after the steps of ablating and/or after the binding of a nucleic acid primer to a nanowire surface, the nanowire surface can be saturated by passivation, preferably with polyethylene glycol derivatives. Preferably, this is followed by a washing step for removing excess passivation reagent or excess nucleic acid.

In a further embodiment, the clonal bridge amplification comprises the following step: hybridizing a single-stranded nucleic acid molecule to a nucleic acid primer, on the surface of a nanowire. Preferably, during the hybridization of a single-stranded nucleic acid molecule to a nucleic acid primer on the nanowire surface, only a single single-stranded nucleic acid molecule is hybridized to a first nucleic acid primer from among a multiplicity of first nucleic acid primers. This has the effect that the first single-stranded nucleic acid molecule is clonally amplified. It is understood that in the case of hybridizing a plurality of single-stranded nucleic acid molecules to a plurality of nucleic acid primers, the single-stranded nucleic acid molecules have a mutually identical nucleotide sequence. In particular embodiments, the single-stranded nucleic acid molecule which hybridizes to the nucleic acid primer is a forward strand. In other particular embodiments of the method, after step (a) for nucleic acid amplification, the single-stranded nucleic acid molecule which hybridizes to a nucleic acid primer is a reverse strand. By way of example, the clonal bridge amplification comprises the following step: hybridizing a single-stranded nucleic acid molecule, preferably a forward strand, to a first nucleic acid primer, on the surface of one nanowire, for example a second nanowire, or hybridizing a single-stranded nucleic acid molecule, preferably a reverse strand, to a second nucleic acid primer, on the surface of one nanowire, for example of a third nanowire. In both particular embodiments, a directed bridge amplification of the complementary strand to the forward strand and a directed bridge amplification of the complementary strand to the reverse strand are performed.

The directed bridge amplification of a complementary strand to the forward strand can be performed on one array of nanowires. The directed bridge amplification of the copied complementary strands of the reverse strand can be performed on another array of nanowires. In alternative embodiments, the directed bridge amplification of a complementary strand to the forward strand and the directed bridge amplification of the copied complementary strand to the reverse strand can also be performed on different nanowires of the same array.

The directed clonal bridge amplification comprises the following step: elongating a nucleic acid primer using a single-stranded nucleic acid molecule as a template, in order to obtain a nucleic acid double strand on a nanowire surface. It is understood that, after primer elongation, the elongated nucleic acid strand is a complementary counter-strand to the single-stranded nucleic acid molecule template. It is possible to elongate a first nucleic acid primer using a single-stranded nucleic acid molecule, in particular a forward strand, as a template on a second nanowire surface, in order to obtain a nucleic acid double strand, or it is possible to elongate a second nucleic acid primer using a single-stranded nucleic acid molecule, in particular a reverse strand, as a template on a third nanowire surface, in order to obtain a nucleic acid double strand.

In a further embodiment, the clonal bridge amplification comprises the following step: denaturing a nucleic acid double strand bound to one nanowire surface, to obtain a first single-stranded nucleic acid molecule and to obtain a second, other single-stranded nucleic acid molecule bound to the nanowire surface, wherein the first single-stranded nucleic acid molecule is removed. In particular denaturing a nucleic acid double strand bound to a second or a third nanowire surface, and removing the single-stranded nucleic acid molecule to obtain another single-stranded nucleic acid molecule which remains bound to the second or third nanowire surface. It is understood that the single-stranded nucleic acid molecule which is removed was previously the template during the synthesis of the nucleic acid double strand. By way of example, the single-stranded nucleic acid molecule which is removed can be a forward strand or a reverse strand.

In a further embodiment, the clonal bridge amplification comprises the following step: aligning a single-stranded nucleic acid molecule bound to the surface of one nanowire and a nucleic acid primer bound to the surface of another, adjacent nanowire, by preferably an electric field.

In a further embodiment, the clonal bridge amplification comprises the following step: hybridizing a single-stranded nucleic acid molecule bound to one nanowire surface to a nucleic acid primer bound to another, adjacent nanowire surface, by the directed non-stochastic formation of a bridge, the bridge beginning at said one nanowire surface and ending at the other, adjacent nanowire surface. The hybridization can be controlled in the method in steps (a) and (b) by the alignment of the single-stranded nucleic acid molecule and the nucleic acid primer by the electric field. This bridge formation is controlled by the dielectric alignment, since the direction of the alignment can be defined depending on where the positive pole or the negative pole is applied to the nanowire array.

The method according to step (a) furthermore provides the following step: elongating a nucleic acid primer bound to a nanowire surface and hybridized to a single-stranded nucleic acid molecule bound to another, adjacent nanowire surface using the single-strand nucleic acid molecule as a template, in order to obtain a nucleic acid double strand. The nucleic acid double strand forms a bridge beginning at one of the nanowire surfaces and ending at the other, adjacent nanowire surface. The elongation begins from the immobilized nucleic acid primer in the direction 3′ to 5′. It is understood that, after primer elongation, the elongated nucleic acid strand is the inverse counter-strand with respect to the single-stranded nucleic acid molecule template.

A further embodiment of step (a) involves performing: denaturing a nucleic acid double strand, to obtain a first single-stranded nucleic acid molecule bound to one nanowire surface and a second, other single-stranded nucleic acid molecule bound to another, adjacent nanowire surface.

The reaction steps mentioned above can be repeated cyclically in order to amplify nucleic acid molecules of the same sequence on one nanowire surface and on the other, adjacent nanowire surface. Clusters of single-stranded nucleic acid molecules having the same nucleotide sequence among one another are obtained as a result. Preferably, a cluster in each case lies on a nanowire.

During the elongation and the amplification in step a) of the method, H+ are liberated during the synthesis of the nucleic acid strands by a polymerase. The liberated H+ form a charge cloud, lead to a change in the pH value and induce a change in the surface potential at the sensor element surface of the FET. It is thus possible to measure the change in the surface potential using the FET. This allows a quantitative determination of the strand density per nanowire. The amplification is ended if the arising of H+ is no longer measurable.

In an optional embodiment, the clonal bridge amplification comprises the following step: measuring the H+ charge cloud in order to determine the active FETs for step (b) of the bridge sequencing of the method.

Step (a) of the method leads to the formation of a multiplicity of clusters on the nanowires, wherein each cluster comprises a multiplicity of nucleic acid molecules having the same nucleotide sequence. In particular, step (a) of the method leads to an array of nanowires comprising clusters of single-stranded nucleic acid molecules, wherein a cluster is in each case situated on a nanowire. By way of example, an array of nanowires comprises at least one first cluster of single-stranded nucleic acid molecules bound to one nanowire surface, preferably a second nanowire surface, and at least another, second cluster of single-stranded nucleic acid molecules bound to another, adjacent nanowire surface, preferably a third nanowire surface. Each cluster contains clonally amplified single-stranded nucleic acids, wherein a cluster is situated on a nanowire. Preferably, only a cluster of nucleic acid molecules having the same nucleotide sequence is situated on a nanowire. Since stochastic cluster formation does not take place, it is possible to assign a cluster specifically to each nanowire. It is understood that colonies with high density can be made available, depending on the distance between the nanowires. The present method makes it possible to distinguish between clusters that were formed by amplification of forward strands and/or reverse strands. In particular, a cluster on one nanowire can contain amplified forward strands and another nanowire can contain amplified reverse strands. The number of single-stranded nucleic acid molecules of the clusters can be controlled by the number of cycles of alignment, hybridization, primer elongation and double strand denaturing. The clusters can be used in the subsequent step (b) of the method.

In one preferred embodiment, step (a) of the method comprises the following steps and wherein step (ii) can optionally be performed before any other step preceding step (vi):
(i) selectively binding a first nucleic acid primer to one nanowire surface, preferably a second nanowire surface;
(ii) selectively binding a second nucleic acid primer to another, adjacent nanowire surface, preferably a third nanowire surface;
(iii) hybridizing a first single-stranded nucleic acid molecule to the first nucleic acid primer on said one nanowire surface, preferably the second nanowire surface;
(iv) elongating the first nucleic acid primer using the first single-stranded nucleic acid molecule as a template, in order to obtain a first nucleic acid double strand on said one nanowire surface, preferably the second nanowire surface;
(v) denaturing the first nucleic acid double strand bound to said one nanowire surface, to obtain a first single-stranded nucleic acid molecule and to obtain a second, other single-stranded nucleic acid molecule bound to said one nanowire surface, wherein the first single-stranded nucleic acid molecule is removed;
(vi) aligning the second single-stranded nucleic acid molecule bound to said one nanowire surface and the second nucleic acid primer bound to the other, adjacent nanowire surface, by an electric field;
(vii) hybridizing the second single-stranded nucleic acid molecule bound to said one nanowire surface to the second nucleic acid primer bound to the other, adjacent nanowire surface, by the directed non-stochastic formation of a bridge;
(viii) elongating the second nucleic acid primer bound to the other, adjacent nanowire surface and hybridized to the second single-stranded nucleic acid molecule, using the second single-stranded nucleic acid molecule as a template, in order to obtain a second nucleic acid double strand;
(ix) denaturing the second nucleic acid double strand to obtain the second single-stranded nucleic acid molecule bound to said one nanowire surface and a third, other single-stranded nucleic acid molecule bound to the other, adjacent nanowire surface;
(x) cyclically repeating steps (vi)-(ix) in order to amplify nucleic acid molecules of the same sequence on said one nanowire surface and on the other, adjacent nanowire surface;
(xi) optionally measuring the H+ charge cloud in order to determine the active FETs for step (b) of the method for sequencing nucleic acid molecules.

In particular further embodiments of the methods, a step of denaturing a double strand preferably takes place with the aid of temperature in the range of approximately 85-105° C., more preferably in the range of 90-100° C., most preferably in the range of 92-95° C. Denaturing of a double strand, depending on the length thereof, can also be achieved by a chaotropic solvent. After the denaturing of the double strand, the template nucleic acid molecule can be removed by washing.

In the method, step (b) comprises a directed bridge sequencing.

It is understood that after ending step (a) and before step (b) of the method, an array of nanowires is made available, wherein the array comprises: at least one first cluster of single-stranded nucleic acid molecules bound to one nanowire surface, preferably a second nanowire surface, and at least another, second cluster of single-stranded nucleic acid molecules bound to another, adjacent nanowire surface, preferably a third nanowire surface.

In embodiments of step (b), single-stranded nucleic acid molecules bound to one nanowire surface are a cluster of single-stranded nucleic acid molecules.

Before the step of binding a nucleic acid primer to a nanowire surface, step (b) comprises the following step: ablating the polymer coating of the nanowire surface by Joule heating.

In embodiments, step (b) comprises the following step: selectively binding nucleic acid primers, preferably second nucleic acid primers, to one nanowire surface, preferably to a first nanowire surface.

A further embodiment comprises selectively binding nucleic acid primer, preferably first nucleic acid primer, to another nanowire surface, not directly adjacent, preferably to a fourth nanowire surface. In preferred embodiments, two or more further nanowires are situated between said one nanowire to which the first nucleic acid primer is selectively bound and the other nanowire, not directly adjacent, to which the second nucleic acid primer is selectively bound.

Preferably, after the steps of ablating and/or after binding a nucleic acid primer to one nanowire surface, the nanowire surface can be saturated by passivation, preferably with polyethylene glycol derivatives. Preferably, this is followed by a washing step for removing excess passivation reagent or excess nucleic acid.

In a further embodiment, step (b) comprises the following step: aligning single-stranded nucleic acid molecules bound to one nanowire surface and nucleic acid primers bound to another, adjacent nanowire surface, by preferably an electric field. By way of example, step (b) comprises the following step: aligning single-stranded nucleic acid molecules, for example first single-stranded nucleic acid molecules, bound to one nanowire surface, preferably a second nanowire surface, and second nucleic acid primers bound to another, adjacent nanowire surface, preferably a first nanowire surface, by preferably an electric field.

In a further embodiment, step (b) comprises the following step: hybridizing single-stranded nucleic acid molecules bound to one nanowire surface to nucleic acid primers bound to another, adjacent nanowire surface, by the directed non-stochastic formation of a bridge, the bridge beginning at said one nanowire surface and ending at the other, adjacent nanowire surface. The hybridization is supported by the alignment in the electric field. By way of example, step (b) comprises the following step: hybridizing first single-stranded nucleic acid molecules bound to one nanowire surface, preferably a second nanowire surface, to second nucleic acid primers bound to another, adjacent nanowire surface, preferably a first nanowire surface, by the directed non-stochastic formation of a bridge.

A field change of the electric field is preferably effected. What is achieved by the field change is that after the single-stranded nucleic acid molecules of one cluster have hybridized to nucleic acid primers by binding, other clusters can be newly aligned.

The method according to step (b) furthermore provides the following step: aligning single-stranded nucleic acid molecules bound to one nanowire surface, preferably a third nanowire surface, and first nucleic acid primers bound to another, adjacent nanowire surface, preferably a fourth nanowire surface, by preferably a field change of the electric field.

When aligning a plurality of clusters bound on different nanowires of the nanowire array, it is possible to control the alignment for forming bridges. When aligning a plurality of clusters bound on different nanowires of the nanowire array, for example if one cluster of single-stranded nucleic acid strands is bound to the surface of one nanowire, and another cluster of single-stranded nucleic acid strands is bound to the surface of another nanowire, the hybridization of the single-stranded nucleic acid strands of the different clusters to nucleic acid primers can be controlled to form a bridge.

One embodiment involves aligning single-stranded nucleic acid strands of two different clusters respectively bound on the surfaces of two different nanowires and two clusters of different nucleic acid primers likewise respectively bound on the surfaces of two other different nanowires by an electric field such that the two different clusters of single-stranded nucleic acid molecules and the two clusters of different nucleic acid primers are aligned in the same direction. In this case, the two different clusters of single-stranded nucleic acids and the two clusters of different nucleic acid primers lie between the positive pole and the negative pole.

Another embodiment involves aligning the clusters of single-stranded nucleic acid strands in different directions. This embodiment involves aligning a first cluster of single-stranded nucleic acids bound to a second nanowire surface and a cluster of first nucleic acid primers bound to a first nanowire surface in one direction and aligning another, second cluster of single-stranded nucleic acids bound to a third nanowire surface and a cluster of other, second nucleic acid primers bound to a fourth nanowire surface in an opposite direction. In this case, the first cluster of single-stranded nucleic acids bound to a second nanowire surface and a cluster of first nucleic acid primers bound to a first nanowire surface lie between the positive pole and the negative pole and the other, second cluster of single-stranded nucleic acids bound to a third nanowire surface and the cluster of other, second nucleic acid primers bound to a fourth nanowire surface lie outside the field of positive pole and negative pole, in particular on the side facing the negative pole.

In further embodiments, in the step of aligning single-stranded nucleic acid strands for hybridizing with nucleic acid primers excess nucleic acid primer can be added, wherein the excess nucleic acid primer hybridizes to complementary nucleic acid sequence regions of the single-stranded nucleic acids of one cluster. This has the advantage that these sequence regions can no longer hybridize to complementary sequence regions of single-stranded nucleic acids of another cluster.

In one embodiment, the method according to step (b) furthermore provides the following step: hybridizing single-stranded nucleic acid molecules bound to one nanowire surface, preferably a third nanowire surface, to first nucleic acid primers bound to another, adjacent nanowire surface, in particular a fourth nanowire surface, by the directed non-stochastic formation of a bridge. The hybridization of the second single-stranded nucleic acid strands to first nucleic acid primers to form a bridge is supported by the changed electric field.

In preferred embodiments, the first single-stranded nucleic acid molecules are forward strands or reverse strands of a nucleic acid molecule and the second single-stranded nucleic acid molecules are copied forward strands or reverse strands of a nucleic acid molecule. These embodiments ensure that both the forward strand and the reverse strand of a nucleic acid molecule can be sequenced on a single array of nanowires in step (b) of the method. By simultaneously reading out the nucleic acid sequence of the forward strand and of the complementary reverse strand of a nucleic acid, it is possible to improve the quality of the sequencing reaction since the sequence information both of the forward strand and of the reverse strand is present and errors can thus be identified more rapidly. This is possible by sequencing from the opposite direction of the nucleic acid molecule (forward and reverse strands or beginning and end of the nucleic acid molecule) toward the center. This simultaneously enables a paired-end sequencing without additional steps using the same chemistry in the method, e.g. for de novo sequencing applications.

In one embodiment, step (b) of the method comprises: elongating nucleic acid primers bound to one nanowire surface and hybridized to single-stranded nucleic acid molecules bound to another, adjacent nanowire surface, using the single-stranded nucleic acid molecules as a template, in order to obtain nucleic acid double strands. During the elongation of the nucleic acid primers, in each case at least one nucleoside triphosphate is incorporated sequentially at the 3′ end of the nucleic acid primer. In particular, the method comprises: elongating second nucleic acid primers bound to one nanowire surface, in particular a first nanowire surface, and hybridized to first single-stranded nucleic acid molecules bound to another, adjacent nanowire surface, in particular a second nanowire surface, using the first single-stranded nucleic acid molecules as a template, in order to obtain first nucleic acid double strands, or elongating first nucleic acid primers bound to one nanowire surface, in particular a fourth nanowire surface, and hybridized to second single-stranded nucleic acid molecules bound to another, adjacent nanowire surface, in particular a third nanowire surface, using the second single-stranded nucleic acid molecules as a template, in order to obtain second nucleic acid double strands.

In one embodiment, the method according to step (b) furthermore provides the following step: detecting the incorporation of one or more known nucleoside triphosphates by measuring an H+ charge cloud above a nanowire FET.

The elongation begins at the immobilized nucleic acid primers in the direction of 3′ to 5′. During elongation, a directed bridge sequencing of uniform nucleic acid clusters is effected by synthesis by pulsed supply of individual nucleotides and electrical signal read-out. The electrical signal read-out comprises measuring a voltage change by forming an H+ charge cloud. As a result of the synthesis of the nucleic acid strand at the bridge formed, H+ are liberated during the incorporation of the nucleotides by the polymerase in direct proximity to the FET and this causes a change in the pH value. FETs exhibit a change in conductivity in reaction to changes in the electric field or potential by virtue of the H+ at the surface. Preferably, the measurement takes place with a long Debye-Hückel length, usually with a buffer concentration of less than 1 mmol. In some embodiments, the measurement can take place complementarily on p- and n-doped nanowires. The measured signal is correlated with the supplied nucleotide or the incorporation of a plurality of identical nucleotides is determined by the signal amplitude.

In one embodiment, the method according to step (b) furthermore provides the following optional step: denaturing nucleic acid double strands to obtain first single-stranded nucleic acid molecules bound to a first nanowire surface and second, other single-stranded nucleic acid molecules bound to another, adjacent nanowire surface. In particular, the denaturing step comprises: denaturing first nucleic acid double strands to obtain third single-stranded nucleic acid molecules bound to a first nanowire surface and first single-stranded nucleic acid molecules bound to a second, other nanowire surface, adjacent to the first nanowire surface, and denaturing second nucleic acid double strands to obtain second single-stranded nucleic acid molecules bound to a third nanowire surface and fourth single-stranded nucleic acid molecules bound to a fourth, other nanowire surface, adjacent to the third nanowire surface.

In one particular embodiment, step (b) of the method comprises the following steps:
selectively binding second nucleic acid primers to one nanowire surface, preferably a first nanowire surface;
selectively binding first nucleic acid primers to one nanowire surface, preferably a fourth nanowire surface;
aligning first single-stranded nucleic acid molecules bound to a second nanowire surface and second nucleic acid primers bound to the other, adjacent first nanowire surface by preferably an electric field;
hybridizing first single-stranded nucleic acid molecules bound to the second nanowire surface to second nucleic acid primers bound to the other, adjacent first nanowire surface, by the directed non-stochastic formation of a bridge;
aligning second single-stranded nucleic acid molecules bound to a third nanowire surface and first nucleic acid primers bound to another, adjacent fourth nanowire surface by preferably a field change of the electric field;
hybridizing second single-stranded nucleic acid molecules bound to the third nanowire surface to first nucleic acid primers bound to the other, adjacent fourth nanowire surface, by the directed non-stochastic formation of a bridge;
elongating second nucleic acid primers bound to a first nanowire surface and hybridized to first single-stranded nucleic acid molecules bound to another, adjacent second nanowire surface, using the first single-stranded nucleic acid molecules as a template, in order to obtain first nucleic acid double strands, and elongating first nucleic acid primers bound to a fourth nanowire surface and hybridized to second single-stranded nucleic acid molecules bound to another, adjacent third nanowire surface, using the second single-stranded nucleic acid molecules as a template, in order to obtain second nucleic acid double strands,
wherein, with elongating the nucleic acid primers, in each case at least one nucleoside triphosphate is incorporated sequentially at the 3′ end of the nucleic acid primers;
detecting the incorporation of known nucleoside triphosphates by measuring the H+ charge cloud above a nanowire FET on or below the nucleic acid bridges formed. Optional denaturing of the nucleic acid double strands to obtain third single-stranded nucleic acid molecules bound to a first nanowire surface, first single-stranded nucleic acid molecules bound to a second nanowire surface, second single-stranded nucleic acid molecules bound to a third nanowire surface and fourth single-stranded nucleic acid molecules bound to a fourth nanowire surface. This has the advantage that the nanowire array can be used again.

It is understood that in other embodiments the step of selectively binding first nucleic acid primers to a fourth nanowire surface can take place in any step that precedes the step of aligning the second single-stranded nucleic acid molecules bound to a third nanowire surface by an electric field.

In one embodiment, the nucleoside triphosphates are selected from the group consisting of ddATP, dATP, ATP, ddGTP, dGTP, GTP, ddTTP, dTTP, TTP, ddUTP, dUTP, UTP, ddCTP, dCTP or CTP.

In particular embodiments of the method, the first nucleic acid primers which bind selectively to a second nanowire surface have the same nucleotide sequence among one another, the second nucleic acid primers which bind selectively to a third nanowire surface having the same nucleotide sequence among one another, the second nucleic acid primers which bind selectively to a first nanowire surface have the same nucleotide sequence among one another and the first nucleic acid primers which bind selectively to a fourth nanowire surface have the same nucleotide sequence among one another. Furthermore in particular embodiments, the first nucleic acid primers which bind selectively to a second nanowire surface can have the same nucleotide sequence as the first nucleic acid primers which bind selectively to a fourth nanowire surface. Furthermore, the second nucleic acid primers which bind selectively to a third nanowire surface can have the same nucleotide sequence as the second nucleic acid primers which bind selectively to a first nanowire surface.

In further embodiments of the method, a single-stranded nucleic acid molecule has a first part of a nucleotide sequence which is identical to a nucleotide sequence of a nucleic acid primer and a third part of a nucleotide sequence which can hybridize with the nucleotide sequence of a nucleic acid primer. Preferably, the first or the third part of the single-stranded nucleic acid molecules respectively lies at the 3′ or 5′ end. In preferred embodiments, the single-stranded nucleic acid molecules comprise at least a third part of a nucleotide sequence which lies between the first and second parts. The third part preferably comprises the nucleotide sequence which is intended to be amplified and/or sequenced. There can also be further parts of a nucleotide sequence in the single-stranded nucleic acid molecule, e.g. a nucleotide sequence which serves as an ID tag that makes it possible to identify a particular nucleic acid molecule or its complementary molecule. The different parts of the single-stranded nucleic acid molecules can be manipulated by methods according to the related art, or be bound to one another by ligation.

In a particular further embodiment of steps (a) and (b) of the method, at least one further nanowire is situated below a bridge formed during the nucleic acid amplification of the nucleic acid sequencing, wherein the nanowire is a nanowire FET situated between at least two semiconductor or metal nanowires. Preferably, the further nanowire FET is a semiconductor nanowire situated between two metal nanowires. The further nanowire FET preferably has no polymer coating. In one preferred embodiment, the nanowire FET preferably has no polymer coating after the nucleic acid primers have been bound on the nanowire surfaces and these nanowire surfaces have been saturated. In preferred embodiments, the polymer coating of the further nanowire FET is ablated. This ablation is necessary in order to enable the electronic read-out by the further nanowire FET.

The polymer coating is removed before the nucleic acid amplification or the nucleic acid sequencing. The detection of nucleic acids having a sequence length of more than 1 kb can be facilitated by virtue of the fact that a further nanowire FET is situated below the bridge.

In all embodiments, the binding of nucleic acid primers to nanowire surfaces, both in step (a) of bridge amplification and in step (b) of bridge sequencing, as described above, means that the surface is selectively functionalized.

The method sequence of selective surface functionalization comprises:
applying a polymer coating, preferably to a chip comprising at least one array of nanowires;
Joule heating of at least one or more nanowires by selective ablation of the polymer coating;
functionalizing the ablated nanowire surface or nanowire surfaces by selectively binding at least one first nucleic acid primer or by binding a plurality of nucleic acid primers.

Selectively means that it is possible to control to which nanowire surface the one or more nucleic acid primers are bound, since it is possible to control selectively beforehand which nanowire surface is ablated. It is understood that a nanowire surface coated with a polymer cannot bind a nucleic acid primer.

The method of selective surface functionalization can furthermore comprise the following step: hybridizing at least one first single-stranded nucleic acid molecule to a first nucleic acid primer bound to a first nanowire surface.

In all embodiments of the method, as described above, the strand length of a first or second single-stranded nucleic acid molecule, in particular of a single-stranded forward strand and of a single-stranded reverse strand, is preferably 0.1-10 kb. The distribution of the strand length can be implemented by an upstream work step.

In further embodiments, after each step of adding a nucleic acid in all embodiments of the method a washing step for eliminating excess nucleic acid is carried out.

After the end of steps (a) and (b) of the method, the chip can be regenerated by chemical cleaning. This has the advantage that the chip can be reused and the material costs can be reduced.

Furthermore, the inventors propose an array of nanowires.

An array of field effect transistor (FET) sensor elements comprises the array of nanowires. In this case, a FET can comprise at least one nanowire and be a nanowire FET, or at least one FET can lie between in each case two nanowires, or at least one nanowire of two nanowire pairs is a FET.

In particular embodiments, the surface of one or more of the nanowires bears no polymer coating. These uncoated nanowires can bind a nucleic acid molecule.

One embodiment provides an array of nanowires, wherein one or more nucleic acids are bound to at least one nanowire surface. One embodiment provides an array of nanowires, wherein a plurality of nucleic acids are bound to a plurality of nanowire surfaces, wherein only nucleic acids of the same nucleotide sequence are situated on a respective nanowire. Preferably, the nucleic acid is a nucleic acid primer, a single-stranded nucleic acid molecule or a double-stranded nucleic acid molecule. In further embodiments, a cluster of nucleic acids is bound to at least one of the nanowire surfaces. It is understood that the nucleic acid molecules which are all bound to a nanowire surface have the same nucleotide sequence among one another.

A further embodiment provides an array of nanowires, wherein at least two nucleic acids are bound to one or more of the nanowires. In preferred embodiments, the nucleic acid molecules are in each case bound to a different nanowire surface. Preferably, the nucleic acids are nucleic acid primers. With further preference, in the case of a plurality of nucleic acid primers, the nucleic acid primers which are bound to a nanowire surface have an identical sequence. Preferably, the nucleic acid primers which are bound to different nanowire surfaces—e.g. if at least one or more first nucleic acid primers are bound to one nanowire surface and at least one or more second, other nucleic acid primers are bound to another nanowire surface—have a mutually complementary or non-identical sequence.

In embodiments, one or more first nucleic acid molecules are bound to a nanowire surface. One or more second, other nucleic acid molecules are single-stranded nucleic acid molecules, wherein the first part of the second single-stranded nucleic acid molecule hybridizes to the first nucleic acid molecule. Preferably, the first nucleic acid molecule is a nucleic acid primer.

In all embodiments, a nucleic acid is selected from the group consisting of a nucleic acid primer, a single-stranded nucleic acid molecule or a nucleic acid double strand.

One particular embodiment provides an array of nanowires, wherein at least two nucleic acid primers are bound to a different nanowire surface in each case and at least one of the nucleic acid primers is hybridized to a single-stranded nucleic acid molecule. These arrays of nanowires can be used in steps (a) and (b) of the method for nucleic acid amplification or nucleic acid sequencing.

A further embodiment provides an array of nanowires, wherein the nucleic acid comprises a nucleic acid double strand, and wherein one end of the nucleic acid double strand is bound to a first nanowire surface and the other end of the nucleic acid double strand is bound to a second, other nanowire surface to form a bridge.

In one embodiment, the array comprises nanowires having a length of less than or equal to 10 000 nm, preferably of less than or equal to 400 nm, preferably of less than or equal to 200 nm, or preferably of 100-400 nm.

The cross section of the nanowire is triangular, quadrilateral, trapezoidally polygonal or round. The nanowires have a primarily rectangular cross section. Preferably, the nanowires have a width of less than or equal to 100 nm, 75 nm, 50 nm, 30 nm or have a width of equal to 10 nm. Ideally, the width of the nanowires is in a range of 10-100 nm, 10-75 nm, 10-50 nm or most preferably 10-30 nm. The sensitivity of the FET rises with 1/r, where r is the wire radius. This shows that by using virtually one-dimensional nanowires having a small diameter and a small radius, the sensitivity is increased compared with two-dimensional systems.

The nanowires preferably have a height of less than or equal to 200 nm, 150 nm, 110 nm and most preferably of less than or equal to 100 nm. Ideally, the height of the nanowires is in a range of 10-200 nm, 50-150 nm, 90-110 nm or most preferably in a range of 95-105 nm.

Preferably, the nanowires have a pitch (center-to-center distance) that is greater than the width of a nanowire. Preferably, the pitch is less than or equal to 3000 nm, 2000 nm, 1500 nm, 1000, 500, 250 nm, preferably of less than or equal to 100 nm. Ideally, the pitch is in a range of 10-3000 nm, 50-2500 nm, 100-2000 nm or most preferably in a range of 200-1000 nm parallel to one another. By the distance between the parallel wires, it is possible to determine the nucleic acid sequence length of the nucleic acid strands to be synthesized, since a bridge that reaches from one nanowire to another nanowire is formed during the bridge amplification.

A FET sensor element comprises at least one nanowire within a nanowire array. An array of FET sensor elements comprises an array of nanowires. By way of example, a nanowire can be a nanowire FET. In this case, the nanowire is a semiconductor nanowire. The surfaces of the nanowires of a nanowire array form the surface of the array of FET sensor elements. Alternatively, at least one FET can lie between in each case two nanowires within an array. It is understood that then at least one semiconductor nanowire lies between at least two metal nanowires and said semiconductor nanowire or nanowires form(s) at least one FET or a plurality of FETs. The surfaces of the semiconductor nanowires between the metal nanowires of a nanowire array form the surface of the array of FET sensor elements. It is understood that the array of nanowires is in contact with a FET or is capacitively coupled to the FET.

Further embodiments provide a chip, wherein the chip comprises one or more arrays of nanowires. In the case of a multiplicity of arrays on a chip, the nanowires can be addressable independently of one another. One array of the multiplicity of arrays can be arranged in an individual reaction chamber. A plurality of arrays of the multiplicity of arrays can be arranged in an individual reaction chamber. A chip can also contain a single reaction chamber containing the multiplicity of arrays.

The chip preferably contains a microfluidic system. The microfluidic system contains a liquid inlet and a liquid outlet. A liquid and/or constituents contained therein are moved in the direction of the arrays via the microfluidic channels by a driving force, in particular by applying pressure, acoustic energy and/or an electric field. The microfluidic system delivers one or more reagents to one or a multiplicity of arrays. The reagents comprise polar solvents, in particular water, or apolar organic solvents, chaotropic solvents for denaturing nucleic acid double strands, nucleic acid molecules, in particular nucleic acid primers, single-stranded or double-stranded nucleic acid molecules, nucleoside triphosphates, a polymerase suitable for amplifying nucleic acid molecules or purification buffers. In particular embodiments, the microfluidic system is designed such that each array is in contact with a different microfluidic channel. In other embodiments, the arrays from the multiplicity of arrays are connected to one another by a single microfluidic channel. The microfluidic system is configured such that the liquid reaches all arrays substantially at the same time. The microfluidic system of one chip can be configured such that the microfluidic system is brought into contact with the microfluidic system of another chip. In another embodiment, the microfluidic system is configured such that the liquid of one chip is not brought into contact with the liquid of another chip. A microfluidic system has the advantage that the use of reagents can be minimized, and that the sequential work steps are processed rapidly.

Chips are produced on wafers. According to the inventors' proposals, a silicon-on-insulator (SOI) wafer is provided. Preferably, the wafer comprises an Si wafer, with further preference a p-type Si wafer. The Si wafer comprises a thin oxide layer. The thin oxide layer is situated on the Si wafer. The thin oxide layer has a thickness of less than or equal to 1 μm. The Si wafer having the thin oxide layer furthermore comprises a thin doped Si layer. The thin doped Si layer is situated on the thin oxide layer. The thin doped Si layer has a thickness of less than or equal to 200 nm, 150 nm, 110 nm and most preferably of less than or equal to 100 nm. A nanowire array is produced lithographically from the Si layer. The Si wafer comprises an SiO2 coating. Preferably, the SiO2 coating has a thickness of less than 1000 nm, preferably less than 500 nm, with further preference a thickness of 400 nm. In the case of the wafer, the wafer die can be coated with a polymer, preferably PTFE. As described in some embodiments, the polymer is ablatable by Joule heating. The polymer layer preferably has a thickness of less than 100 nm, preferably of less than 50 nm, particularly preferably of less than 40 nm. In one particular embodiment, the polymer coating has a thickness of 30 nm.

The wafer is cut into individual dies. A die is packaged with a disposable polycarbonate flow cell in order to isolate the liquid of the microfluidic system from the electronics. This enables sample loading and electrical and fluidic interfaces in order to enable the amplification reagents and sequencing methods.

In embodiments, the chip contains a die, a housing, connection pins, an interface to one or more nanowire arrays and the sensor and evaluation electronics.

Further embodiments provide a device comprising one or more chips. The chips of the device can be capacitively coupled. The device preferably contains one more microfluidic systems and a liquid supply module. In one embodiment, the microfluidic system of the device is configured such that the microfluidic systems of at least two chips can be in contact with one another. In another embodiment, the microfluidic systems of the chips in the device are not in contact with one another. In this case, the reactions that proceed on the different chips can proceed independently of one another.

A further embodiment provides a kit for sequencing nucleic acid molecules, comprising one or more arrays of nanowires, one or more chips or a device and one or more amplifying or sequencing reagents.

Amplifying and sequencing reagents are reagents required for carrying out the bridge amplification or the bridge sequencing according to steps (a) and (b) of the method. The reagents comprise polar solvents, in particular water, or apolar organic solvents, chaotropic solvents for denaturing nucleic acid double strands, nucleic acid molecules, in particular nucleic acid primers, single-stranded or double-stranded nucleic acid molecules, nucleoside triphosphates, a polymerase suitable for amplifying nucleic acid molecules or purification buffers. Furthermore, a kit comprises instructions for the use of one or more arrays of nanowires, of one or more chips or of a device and of amplifying or sequencing reagents according to the method. Alternatively, amplifying or sequencing reagents can be packaged separately.

According to the proposals, the array, the FET, the SOI wafer and the device can be used for sequencing nucleic acid molecules. In particular, they can be used for amplifying and sequencing nucleic acid molecules. One particular design is the use of the array, of the chip or of the device for analyzing nucleic acid molecules.

The nucleic acid molecules originate from one or from a plurality of different samples. The nucleic acid molecules of the first sample comprise a nucleotide sequence which is not identical to the nucleotide sequence of the second sample. Particularly in the case of a multiplicity of samples, the nucleic acid molecules of one sample comprise a nucleotide sequence which is not identical to the nucleotide sequence of a nucleic acid molecule of some other sample.

A sample comprises, for example, a blood sample, a tissue sample, a saliva sample, a urine sample, a stool sample, a soil sample or a water sample or the like.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 shows a nanowire array after the lithographic patterning of an SOI wafer;

FIGS. 2 to 23 illustrate the method by bridge amplification, wherein the following individual steps are shown as follows:

FIG. 2 shows a cross section through a wafer die or a chip coated with a polymer;

FIG. 3 shows a nanowire FET or metal nanowire exposed by ablation of the polymer;

FIG. 4 shows the binding of a nucleic acid primer to an ablated nanowire surface;

FIG. 5 shows the saturation of a metallic or semiconductor surface of a nanowire by passivation;

FIG. 6 shows the ablation of the polymer on a nanowire surface of another, second nanowire FET or metal nanowire;

FIG. 7 shows the binding of a nucleic acid primer to the ablated nanowire surface of another nanowire;

FIG. 8 shows the saturation of the metallic or semiconductor surface of another nanowire by passivation;

FIG. 9 shows the hybridization of a single-stranded forward strand of a nucleic acid molecule to a nucleic acid primer on a nanowire surface;

FIG. 10 shows the hybridization of a single-stranded reverse strand of a nucleic acid molecule to a nucleic acid primer on a nanowire surface;

FIG. 11 and FIG. 12 in each case show the state after the elongation of a nucleic acid primer for the synthesis of a double strand on a nanowire surface;

FIG. 13 and FIG. 14 in each case show the denaturation products of a nucleic acid double strand;

FIG. 15 and FIG. 16 in each case show the alignment of a single-stranded nucleic acid strand and of a nucleic acid primer by an electric field;

FIG. 17 and FIG. 18 in each case show the hybridization of a single-stranded nucleic acid molecule immobilized on one nanowire surface to a nucleic acid primer immobilized on another, adjacent nanowire surface, by directed, non-stochastic formation of a bridge;

FIG. 19 and FIG. 20 in each case show the elongation of a nucleic acid primer for the synthesis of a double strand beginning on the nanowire surface on which the nucleic acid primer is immobilized in the direction of another, adjacent nanowire;

FIG. 21 and FIG. 22 in each case show a state after the denaturation of a nucleic acid double strand to obtain a first nucleic acid single strand on one nanowire surface and another, second nucleic acid single strand on another, adjacent nanowire surface;

FIG. 23 shows two amplified nucleic acid clusters immobilized on nanowire surfaces.

FIGS. 24 to 41 illustrate the method by bridge sequencing, wherein the following individual steps are shown as follows:

FIG. 24 shows the ablation of the polymer on a nanowire surface;

FIG. 25 shows the binding of a nucleic acid primer to an ablated nanowire surface and the hybridization of excess nucleic acid primers to complementary nucleic acid sequence regions of the single-stranded nucleic acid molecules of a cluster;

FIG. 26 shows the denaturation and elimination of excess nucleic acid primers and the saturation of a nanowire surface by passivation;

FIG. 27 shows the ablation of the polymer on a nanowire surface;

FIG. 28 shows the binding of a nucleic acid primer to an ablated nanowire surface and the hybridization of excess nucleic acid primers to complementary nucleic acid sequence regions of a cluster of single-stranded nucleic acid molecules;

FIG. 29 shows the denaturation and elimination of excess nucleic acid primers and the saturation of a nanowire surface by passivation;

FIG. 30 shows the alignment of two different clusters of single-stranded nucleic acid molecules respectively bound on the surfaces of two different nanowires and of two clusters of different nucleic acid primers likewise respectively bound on the surfaces of different nanowires, by an electric field, wherein the two different clusters of single-stranded nucleic acid molecules and the two clusters of different nucleic acid primers are aligned in the same direction;

FIG. 31 shows the alignment of single-stranded nucleic acid strands of two different clusters respectively bound on the surfaces of two different nanowires and of two clusters of different nucleic acid primers likewise respectively bound on the surfaces of different nanowires, by an electric field in opposite directions and the addition and hybridization of excess nucleic acid primers to complementary nucleic acid sequence regions of one cluster;

FIG. 32 shows the denaturation and elimination of excess nucleic acid primers and the opposite alignment of the nucleic acid strands in the electric field;

FIG. 33 shows the hybridization of single-stranded nucleic acid molecules immobilized on one nanowire surface to nucleic acid primers immobilized on another, adjacent nanowire surface, by directed, non-stochastic formation of a bridge;

FIG. 34 shows the hybridization of single-stranded nucleic acid molecules immobilized on one nanowire surface to nucleic acid primers immobilized on another, adjacent nanowire surface, by directed, non-stochastic formation of a bridge with addition and hybridization of excess nucleic acid primers to complementary nucleic acid sequence regions of one cluster;

FIG. 35 shows the hybridization of single-stranded nucleic acid molecules immobilized on one nanowire surface to nucleic acid primers immobilized on another, adjacent nanowire surface, by directed, non-stochastic formation of a bridge and application of a positive charge to one nanowire and a negative charge to another nanowire;

FIG. 36 shows the hybridization of single-stranded nucleic acid molecules immobilized on one nanowire surface to nucleic acid primers immobilized on another, adjacent nanowire surface, by directed, non-stochastic formation of a bridge and the alignment of other non-hybridized single-stranded nucleic acid strands and other nucleic acid primers by an electric field;

FIG. 37 shows the hybridization of single-stranded nucleic acid molecules immobilized on nanowire surfaces to nucleic acid primers immobilized on other, adjacent nanowire surfaces, by directed, non-stochastic formation of a bridge;

FIG. 38 shows the elongation of nucleic acid primers for the synthesis of double strands beginning on the nanowire surface on which the nucleic acid primers are immobilized in the direction of another, adjacent nanowire;

FIG. 39 shows the arising of a charge cloud composed of H+ which arises as a result of the incorporation of nucleotides, during the sequencing by synthesis, by a polymerase;

FIG. 40 shows one embodiment, wherein in this embodiment a further nanowire is situated below a nucleic acid bridge and between two nanowires, wherein the two nanowires are connected by a nucleic acid bridge;

FIG. 41 shows nucleic acid double strands after a directed bridge sequence;

FIG. 42 shows a chip after removal of the microfluidic assembly and regeneration of the chip by chemical cleaning.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

The accompanying drawings are intended to illustrate embodiments of the proposals and to convey a further understanding thereof. The nucleotide sequences in the figures merely serve the purpose of elucidating the principles of the method and are not intended to be interpreted as being restricted to the nucleotide sequences specified. The nucleotide sequences can be replaced by other nucleotide sequences, which can also be longer or shorter. In connection with the description they serve for clarifying concepts and principles. Other embodiments and many of the advantages mentioned are evident with regard to the drawings. The elements in the drawings are not necessarily illustrated as true to scale with respect to one another. Identical, functionally identical and identically acting elements, features and components are in each case provided with the same reference signs in the figures of the drawings, unless explained otherwise.

A DNA containing a nucleotide sequence to be sequenced is split into random fragments by mechanical shear forces that develop during disintegration of the DNA or by other methods. The fragment containing the nucleotide sequence to be sequenced is ligated to ID tags (identification tags) as flanking elements at the 3′ and/or 5′ end. The product obtained is subsequently ligated to forward and reverse adapters at the 3′ and 5′ end in order to obtain a ligation product. The ligation product is used in the further course of the method. In order to obtain a higher integration density, length selection can be carried out prior to the sequencing. Alternatively, if a low integration density is present, the length selection can take place on the chip.

An SOI wafer is lithographically patterned in order to obtain chips on the wafer.

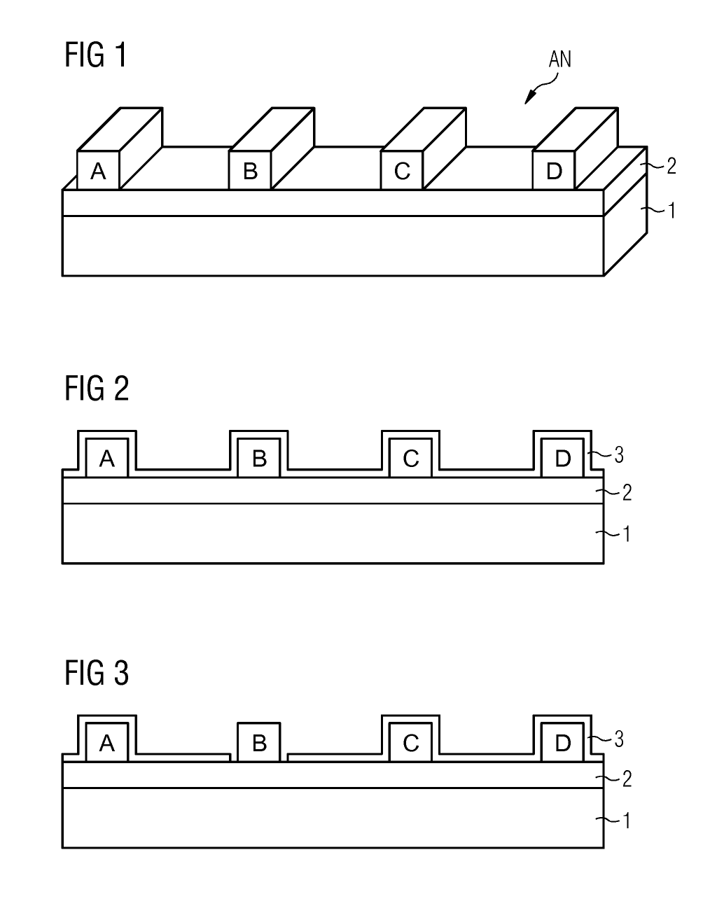

FIG. 1 shows a nanowire array AN comprising in each case four nanowires A, B, C, D. The nanowire array AN and ISFETs are contained on the chip. The nanowires A, B, C, D are situated on an SiO2 layer 2. The SiO2 layer is situated on a silicon layer 1.

Three different FET architectures are realized during the lithographic patterning. In a first variant, a FET contains a nanowire (nanowire FET) A, B, C or D, which can be p-type, n-type or complementary, having typical dimensions of 10-100 nm diameter and lengths of 100-10 000 nm. The nanowires are parallel to one another at a distance of 100-2000 nm. In a second variant, the nanowires A, B, C or D are metallic nanowires and not FET nanowires. For the electronic read-out, therefore, FETs lie between the metal nanowires A-B and C-D. In a third variant, in each case one nanowire of the pairs A-B or C-D is a nanowire FET.

FIG. 2 shows the coating of a wafer die or of a chip with a polymer 3, e.g. PTFE. The polymer is ablatable by Joule heating.

The directed bridge amplification of a forward strand on a nanowire array having the nanowire surfaces A, B, C or D and the directed bridge amplification of a reverse strand on a nanowire array having the nanowire surfaces A′, B′, C′ or D′ is described below.

FIG. 3 shows the exposure of a nanowire FET or metal nanowire B by ablation of the polymer 3. The ablation is effected by Joule heating, thereby exposing the nanowire FET metal nanowire surface B. This involves using temperatures of up to 300° C. with approximately 2.5 V/μm nanowire length, with a nanowire width and a nanowire height of 100×100 nm. The lateral resolution of the polymer layer around the nanowire by this process is less than 200 nm given a temperature decrease of approximately ˜200° C./200 nm.

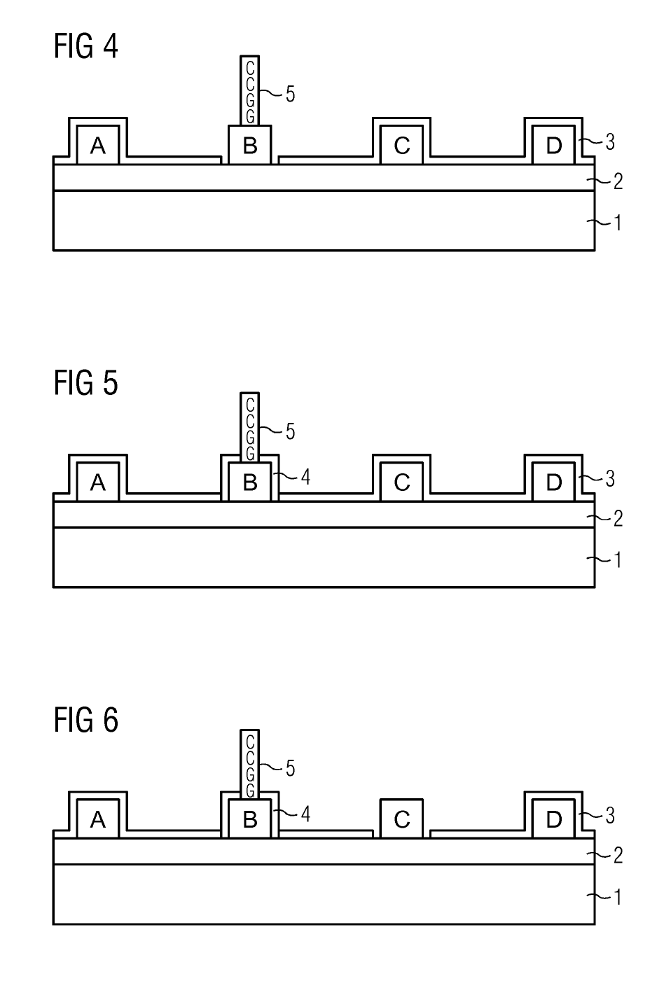

FIG. 4 shows the binding of a DNA primer 5 to the nanowire FET surface B exposed by ablation. The binding can be effected by a covalent bond. A washing step is carried out in order to eliminate excess DNA primers. All work steps are carried out in a microfluidic environment in order to minimize the use of reagents and in order to rapidly process the sequential work steps.

FIG. 5 shows the additional saturation of the metallic or semiconductor surfaces B to which the DNA primer 5 is bound by passivation. A passivation layer 4 is formed. By way of example, the passivation is effected with PEG derivatives. In order to remove excess passivation reagent, a washing step is carried out.

FIG. 6 shows the exposure of a second nanowire FET or metal nanowire C by ablation of the polymer 3 by Joule heating. The same reaction conditions as for the ablation of nanowire surface B are used in this case.

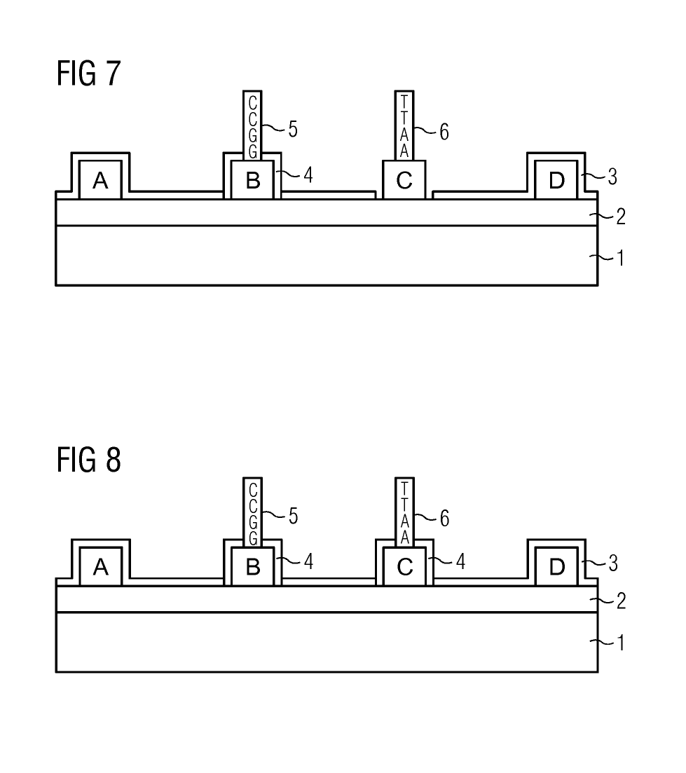

As shown in FIG. 7, a DNA primer 6 is bound to the ablated nanowire surface C by covalent binding. A subsequent washing step is carried out in order to eliminate excess DNA primers.

FIG. 8 shows the additional saturation of the metallic or semiconductor surfaces C to which the DNA primer 6 is bound by passivation. A passivation layer 4 is formed. By way of example, the passivation is effected with PEG derivatives. In order to remove excess passivation reagent, a washing step is carried out.

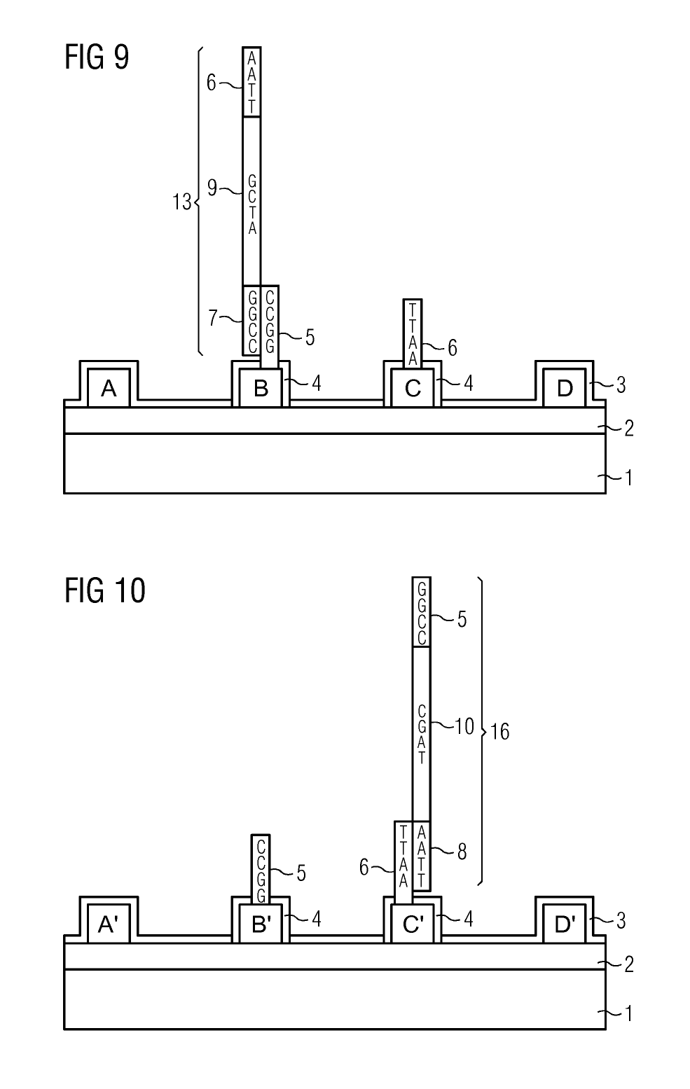

FIG. 9 shows the hybridization of a single-stranded forward strand of a DNA molecule 13 to the DNA primer 5 on the nanowire surface B.

FIG. 10 shows the hybridization of a single-stranded reverse strand of a DNA molecule 16 to a DNA primer 6 on the nanowire surface C′. Although the method explained below are explained on two different nanowire arrays, it is understood that these separately explained steps can also be carried out on different nanowires of a single nanowire array. Ideally, only a single single-stranded DNA 13 hybridizes to the DNA primer 5 on the nanowire surface B and a single single-stranded DNA 16 hybridizes to the DNA primer 6 on the nanowire surface C′. It is understood that with optimum dilution a plurality of single-stranded DNA molecules 13 and respectively 16 also bind. The single-stranded DNA 13 is a forward strand of a DNA and the single-stranded DNA 16 is a reverse strand of the DNA. The sequence of the adapter sequence 7 of the single-stranded DNA 13 is complementary to the nucleotide sequence of the DNA primer 5 and the sequence of the adapter sequence 8 of the single-stranded DNA 16 is complementary to the nucleotide sequence of the DNA primer 6. The single-stranded DNA molecule 13 has a target sequence 9 and an adapter sequence 6. The single-stranded DNA molecule 16 has a target sequence 10 and an adapter sequence 5. The forward strand 13 and the reverse strand 16 of the DNA are in each case the initially prepared ligation product, as described above. By using a correspondingly diluted reaction batch of the single-stranded DNAs 13 and 16, the intention is to achieve single-strand binding on the nanowires B and C′. Extrapolated to a nanowire array comprising in each case four nanowires, this means statistically an occupation density of typically ˜37% of the nanowires with a single-stranded DNA. The strand length of the single-stranded nucleic acid is 0.1-10 kb. The distribution of the strand length can be implemented by an upstream work step. Generally, the strand length of the single-stranded nucleic acid is sufficient to form a bridge from one nanowire to another nanowire, that is to say that the strand length must correspond at least to the pitch.

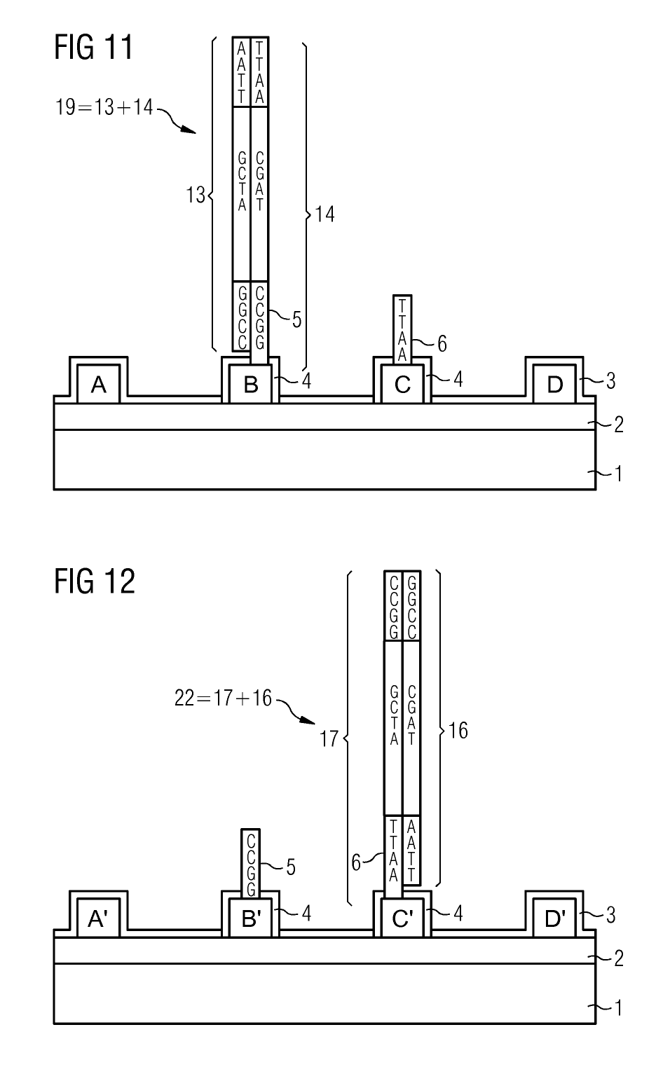

As shown in FIG. 11 the DNA double strand 19 and as shown in FIG. 12 the DNA double strand 22 are synthesized by addition of highly processive DNA polymerase and unmarked NTPs in a buffer suspension, typically in the micromolar or millimolar range, on the nanowire surfaces B and C′, respectively, wherein the synthesis of the two DNA single strands 14 and 17, respectively, which are complementary to the hybridized forward strands 13 and 16, respectively, begins on the immobilized DNA primers 5 and 6, respectively. The DNA primers 5 and 6 are elongated.

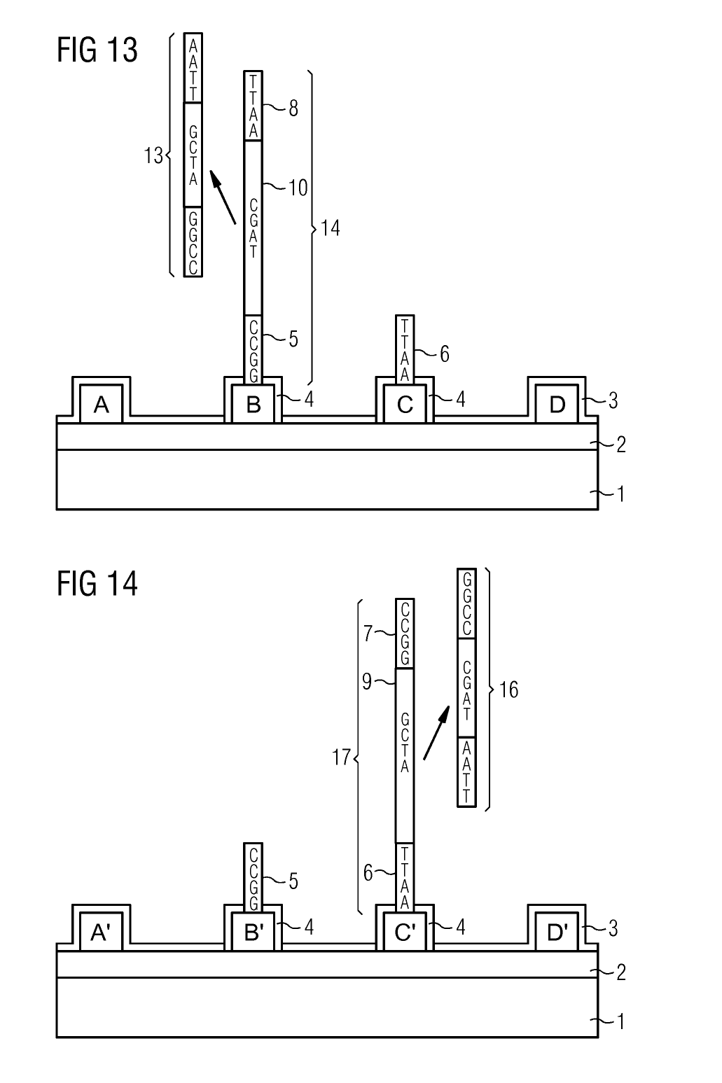

FIG. 13 and FIG. 14 in each case show the denaturation products from the nucleic acid double strands 19 and 22. The DNA double strands 19 and 22 are denatured with the aid of temperatures of between approximately 92-95° C. to obtain a single-stranded DNA molecule 14 on nanowire surface B and a single-stranded DNA molecule 17 on nanowire surface C′. This is effected by a Peltier element or Joule heating. The single-stranded DNA templates 13 and 16 are eliminated by washing. The denaturing of the DNA double strands 19 and 22, depending on their length, can also be achieved by a chaotropic solvent.

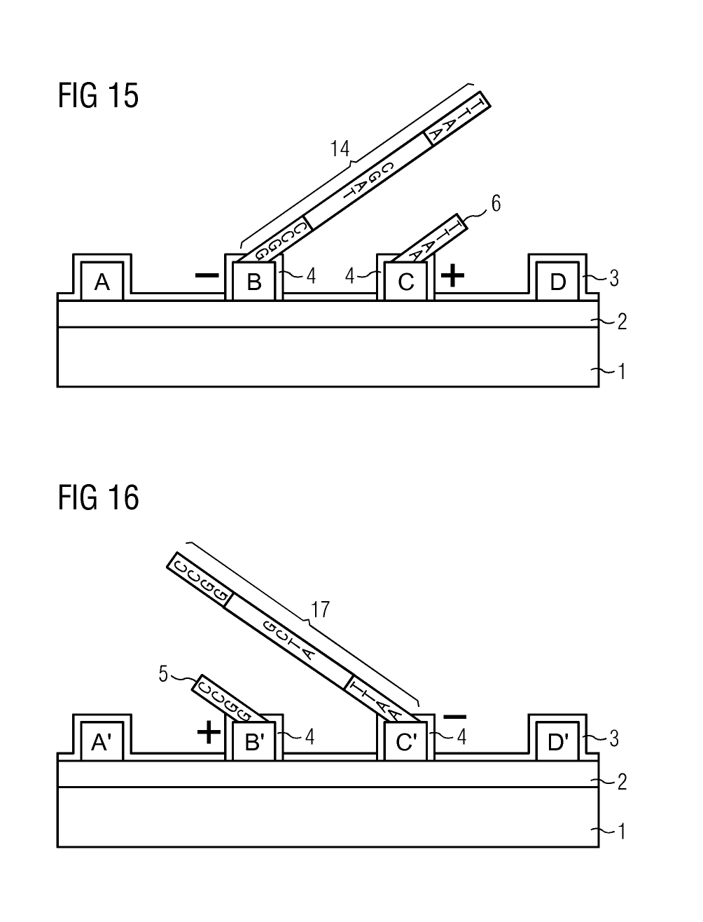

FIG. 15 and FIG. 16 in each case show the alignment of a single-stranded nucleic acid strand and of a nucleic acid primer by an electric field. The single-stranded DNA strand 14 bound to the nanowire surface B and the nucleic acid primer 6 bound to the nanowire surface C are aligned by the electric field. The symbols (+) and (−) indicate the poles of the electric field. As shown in FIG. 16, the single-stranded DNA strand 17 bound to the nanowire surface C′ and the nucleic acid primer 5 bound to the nanowire surface B′ are aligned.

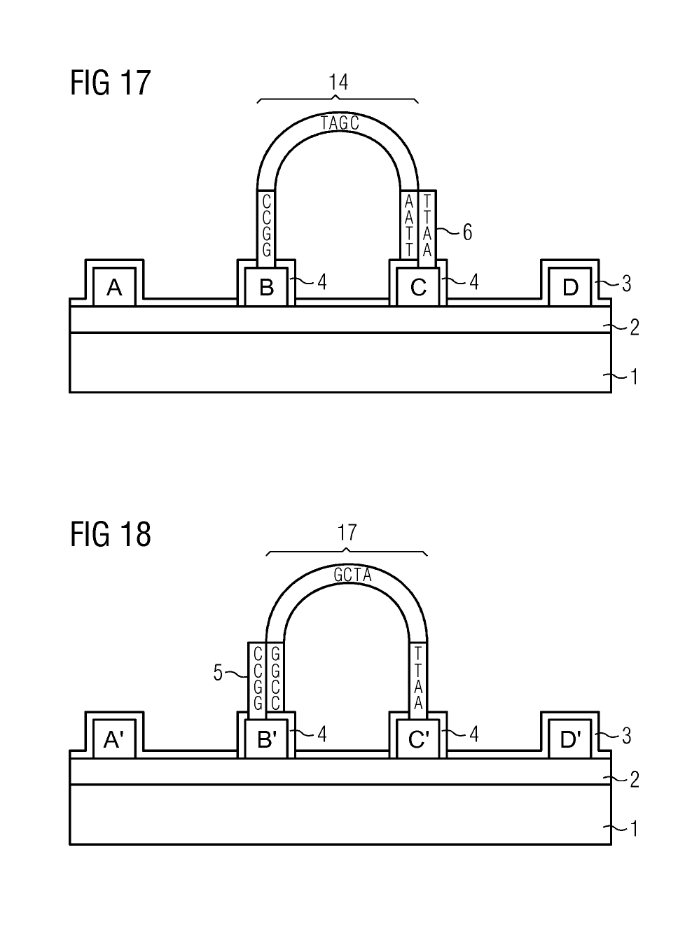

In accordance with the illustrations in FIG. 17 and FIG. 18: the DNA strand 14 immobilized on the nanowire surface B hybridizes to the DNA primer 6 on the nanowire surface C and the DNA strand 17′ immobilized on the nanowire surface C′ hybridizes to the DNA primer 5 on the nanowire surface B′ by directed non-stochastic formation of a bridge. This bridge formation is controlled by the dielectric alignment, since the direction of the alignment can be defined depending on where the positive or negative pole is applied to the nanowire array.

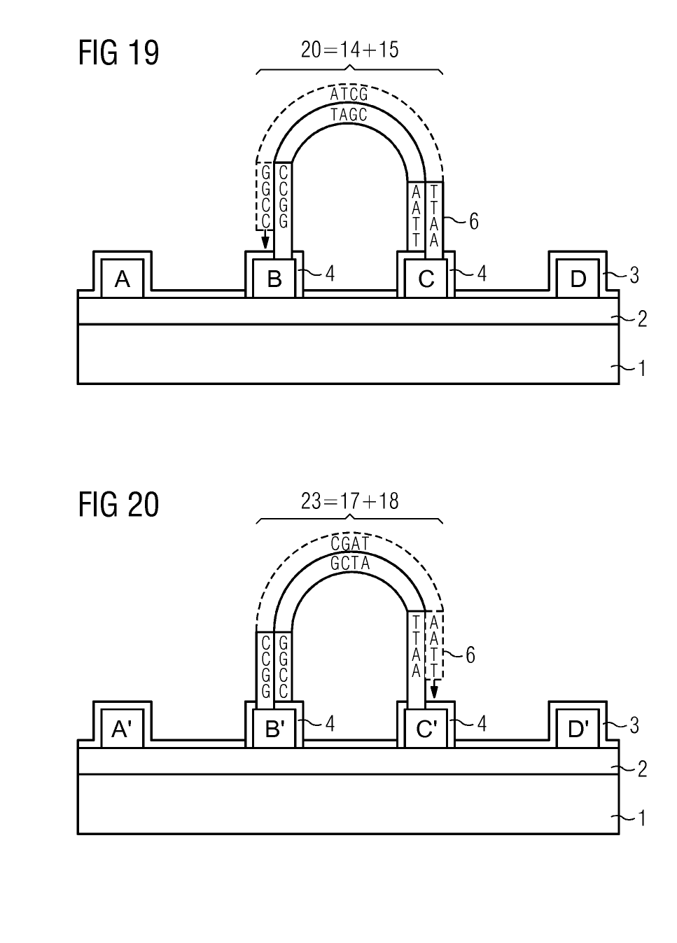

FIG. 19 and FIG. 20 in each case show the elongation of a nucleic acid primer for the synthesis of a double strand beginning on the nanowire surface on which the nucleic acid primer is immobilized in the direction of another, adjacent nanowire; the DNA primer 6 is elongated using the single-stranded DNA molecule 14 as a template with respect to a DNA single strand 15, in order to obtain a DNA double strand 20, and the DNA primer 5 is elongated using the single-stranded nucleic acid molecule 17 as a template with respect to a DNA single strand 18, in order to obtain a DNA double strand 23. The DNA double strands 20 and 23 are synthesized beginning from the immobilized DNA primers on nanowire surfaces C or B′ in the direction of B or C′, 3′ to 5′.

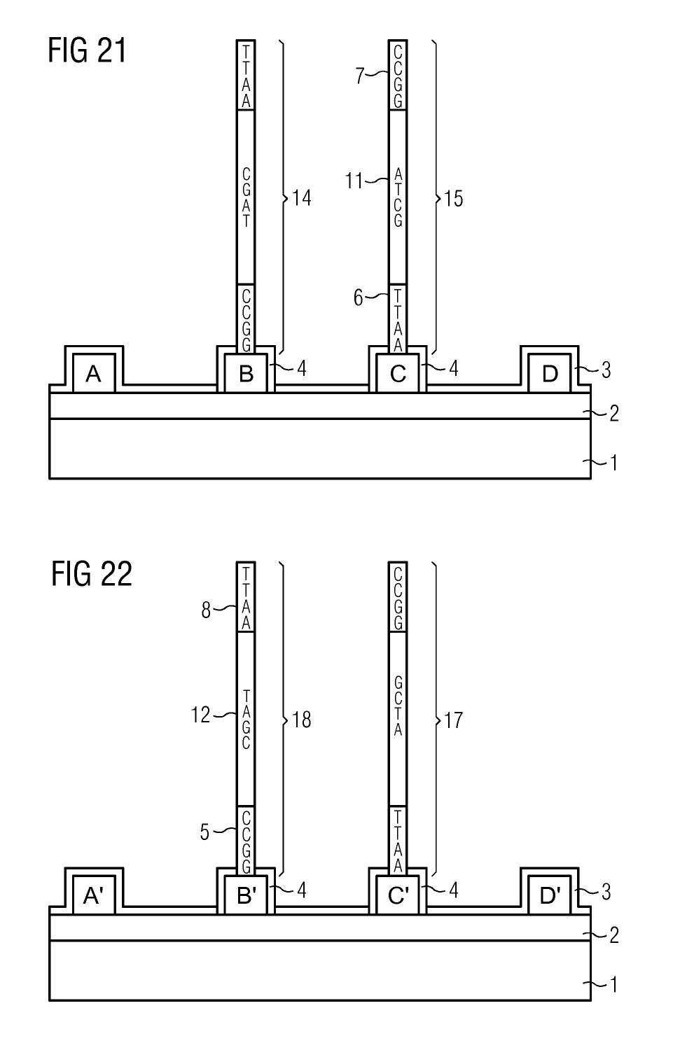

The covalently bonded DNA double strands 20 and 23 are melted and denatured either by temperatures in the range of approximately 92-95° C. or by a chaotropic solvent. FIG. 21 and FIG. 22 in each case show a state after the denaturing of the two DNA double strands. DNA double strand 20 is denatured such that the DNA single strand 14 is obtained on the nanowire B and the DNA single strand 15 is obtained on the nanowire C. DNA double strand 23 is denatured such that the DNA single strand 18 is obtained on the nanowire B′ and the DNA single strand 17 is obtained on the nanowire C′.

By cyclically repeating the abovementioned reaction steps, namely aligning a single-stranded DNA molecule and a DNA primer, hybridizing the single-stranded DNA molecule to the DNA primer by the directed non-stochastic formation of a bridge; elongating the DNA primer using the single-stranded DNA molecule as a template, in order to obtain a DNA double strand, and denaturing the DNA double strand to obtain two single-stranded nucleic acid molecules, a high occupation density of one DNA strand type per nanowire is achieved and clonal clusters of amplified DNA molecules of the same nucleotide sequence are formed on a nanowire surface.

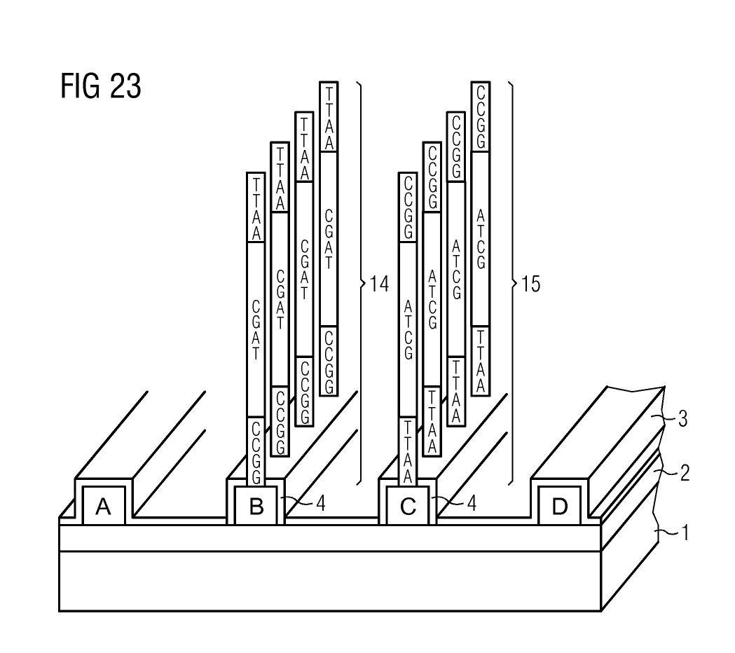

FIG. 23 shows two amplified nucleic acid clusters. After the directed clonal bridge amplification has ended, a cluster of single-stranded DNA molecules of the same sequence 14 is situated on nanowire B and a cluster of single-stranded DNA molecules of the same sequence 15 is situated on nanowire C, wherein the single-stranded DNA molecules of the two clusters are immobilized on the nanowire surfaces.

The H+ liberated during the synthesis of the counter-strands by the DNA polymerase will be measured by the nanowire FETs, which allows a quantitative determination of the strand density per nanowire. The amplification is ended if the arising of H+ is no longer measurable.

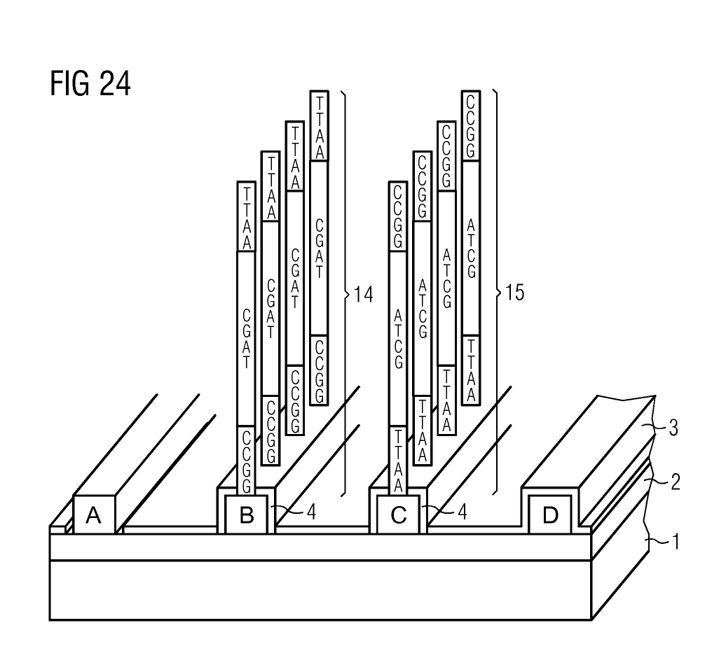

In accordance with the illustration in FIG. 24, a first nanowire surface A is exposed by Joule heating. The same reaction conditions for the ablation as described above are used in this case.

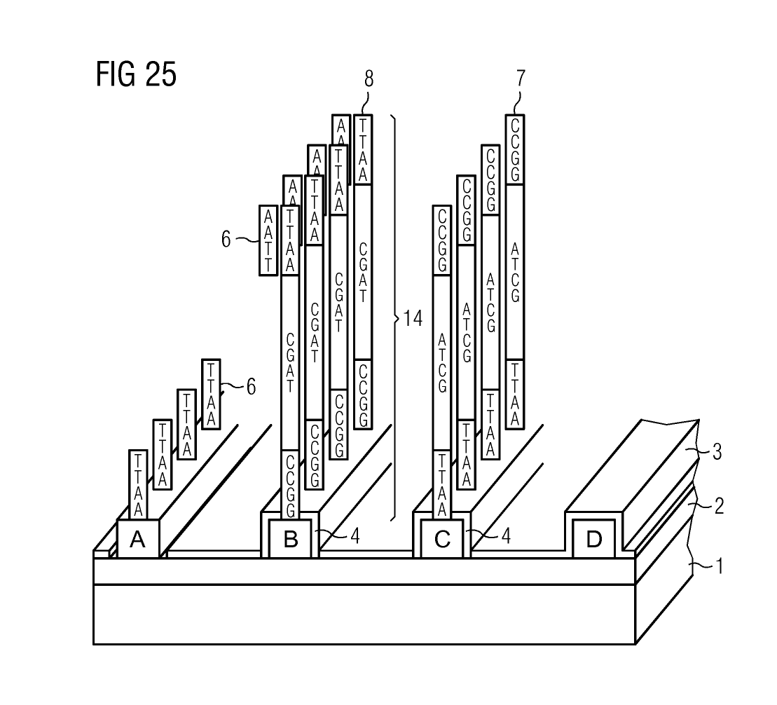

FIG. 25 shows the covalent bonding of DNA primers 6 to the nanowire surface A. Since an excess of DNA primer 6 is used, excess DNA primers 6 hybridize to complementary nucleic acid sequence regions 8 of the single-stranded DNA molecules 14 of the cluster.

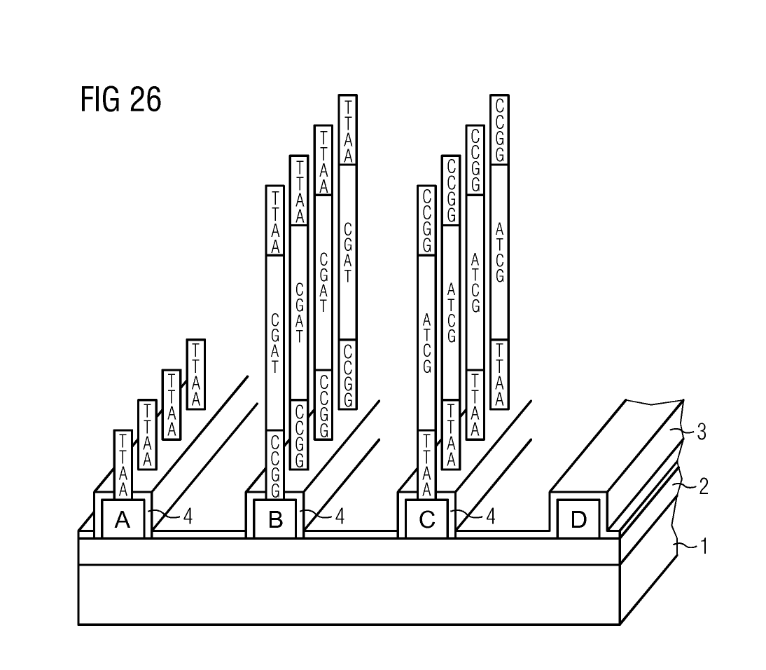

In order to eliminate excess DNA primers 6, as shown in FIG. 26, a washing step is carried out after the denaturing. An additional saturation of the nanowire surface A with e.g. PEG derivatives is effected by passivation. A passivation layer 4 is formed. In order to remove excess passivation reagent, a washing step is carried out again.

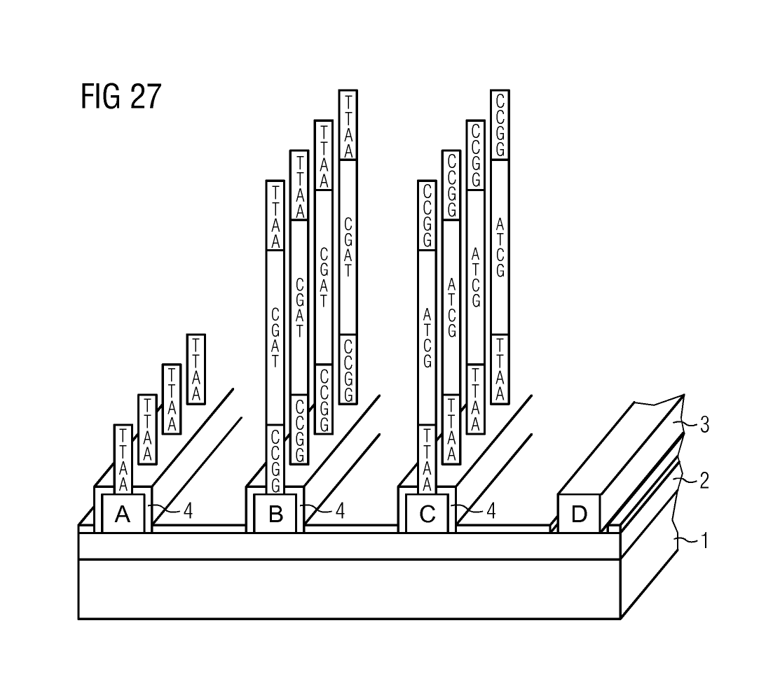

FIG. 27 shows the ablation of the polymer 3 by Joule heating on the nanowire surface D. The same reaction conditions for the ablation as described above are used in this case.

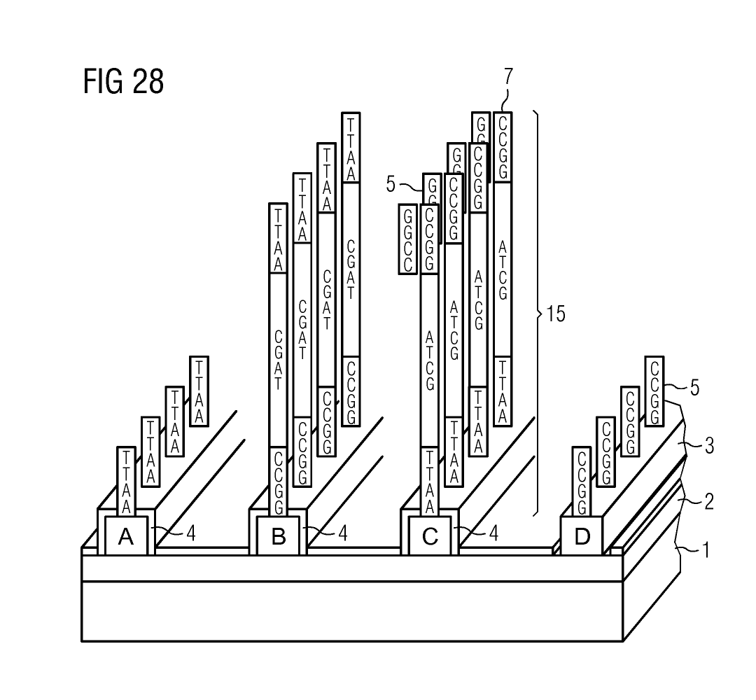

FIG. 28 shows the covalent bonding of DNA primers 5 to the nanowire surface D. Since an excess of DNA primer 5 is used, excess DNA primers 5 hybridize to complementary nucleic acid sequence regions 7 with the single-stranded DNA molecules 15 of the cluster.

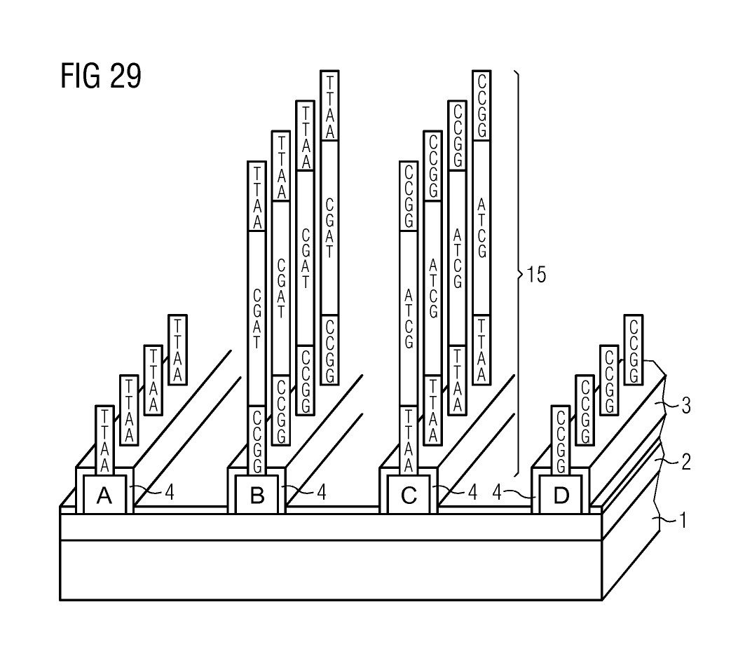

In order to eliminate excess DNA primers 5, as shown in FIG. 29, a washing step is carried out after the denaturing. An additional saturation of the nanowire surface D with e.g. PEG derivatives is effected by passivation. A passivation layer 4 is formed. In order to remove excess passivation reagent, a washing step is carried out again.

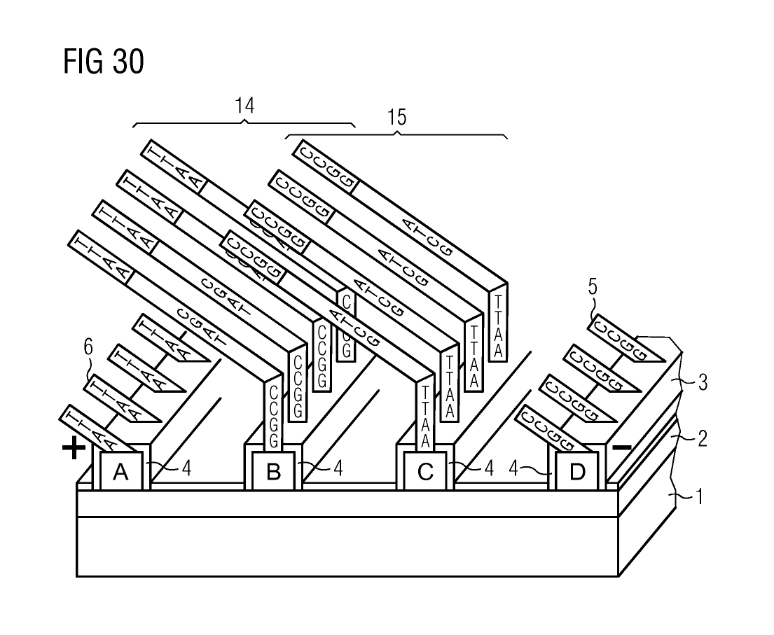

FIG. 30 shows the alignment of single-stranded DNA molecules and DNA primers. In this case, by applying an electric field, firstly the single-stranded DNA molecules 14 of one DNA cluster, the single-stranded DNA molecules 15 of the other cluster, which are in each case bound on the surfaces of two different nanowires, and the DNA primers 6 and the DNA primers 5, which are likewise in each case bound on the surfaces of different nanowires, are aligned in the same direction. This is achieved by virtue of the fact that the nanowires to which both the two clusters of single-stranded DNA molecules 14 and 15 and the DNA primers 6 and 5 are bound lie between the positive pole and the negative pole of the electric field.

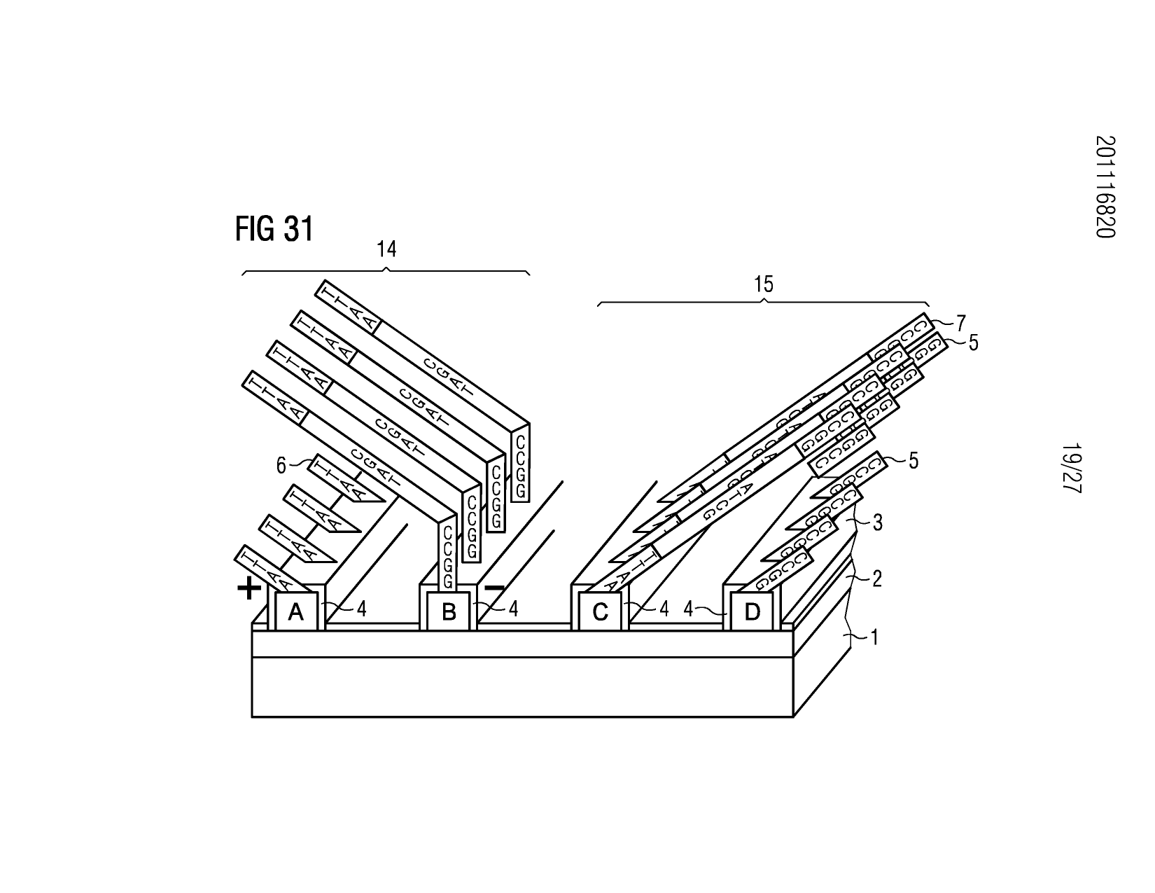

FIG. 31 shows another embodiment of the step of alignment to an electric field, wherein the single-stranded DNA molecules of the clusters are in each case aligned in different directions. In this embodiment, a first cluster of single-stranded DNA molecules 14 bound to the nanowire surface B and a cluster of nucleic acid primers 6 bound to the nanowire surface A are aligned in one direction and another cluster of single-stranded nucleic acids 15 bound to a nanowire surface C and a cluster of nucleic acid primers 5 bound to a nanowire surface D are aligned in an opposite direction. In this case, the first cluster of single-stranded DNA molecules 14 bound to the nanowire surface B and the nucleic acid primers 6 bound to the nanowire surface A lie between the positive pole and the negative pole, and the other cluster of single-stranded DNA molecules 15 bound to the nanowire surface C and the nucleic acid primers 5 bound to the nanowire surface D lie outside the field of positive pole and negative pole, in particular on the side facing the negative pole. This last brings about a repulsion reaction of the negatively charged DNA molecules by the negative pole. In order to prevent sequence regions 7 of the single-stranded DNA molecules 15 from hybridizing to complementary sequence regions 5 of single-stranded DNA molecules 14, an excess of DNA primer 5 is added which hybridizes to complementary nucleic acid sequence regions 7 of the single-stranded DNA molecules 14.

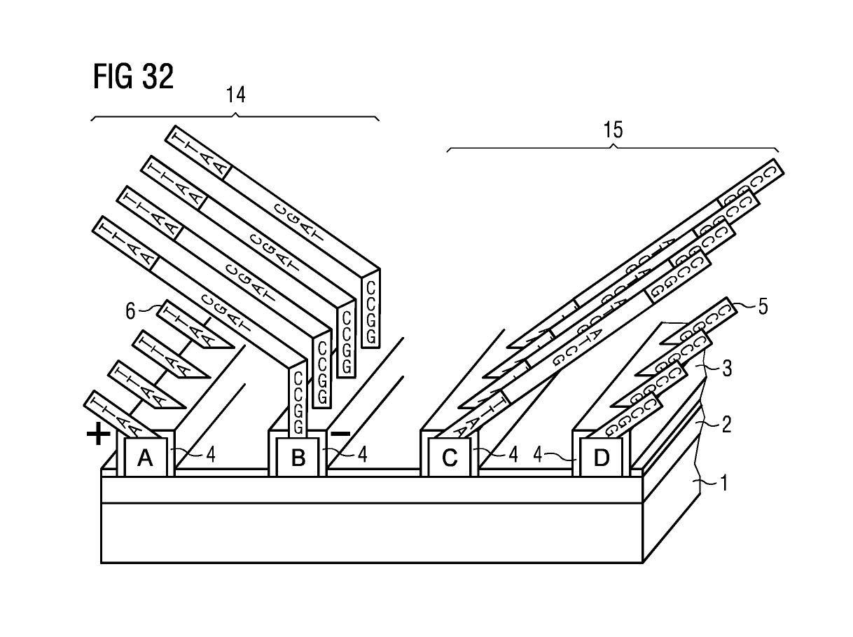

FIG. 32 shows the denaturation and elimination of excess nucleic acid primers 5 and the opposite alignment of the nucleic acid strands 14 and 15, and respectively of the nucleic acid primers 6 and 5, in the electric field.

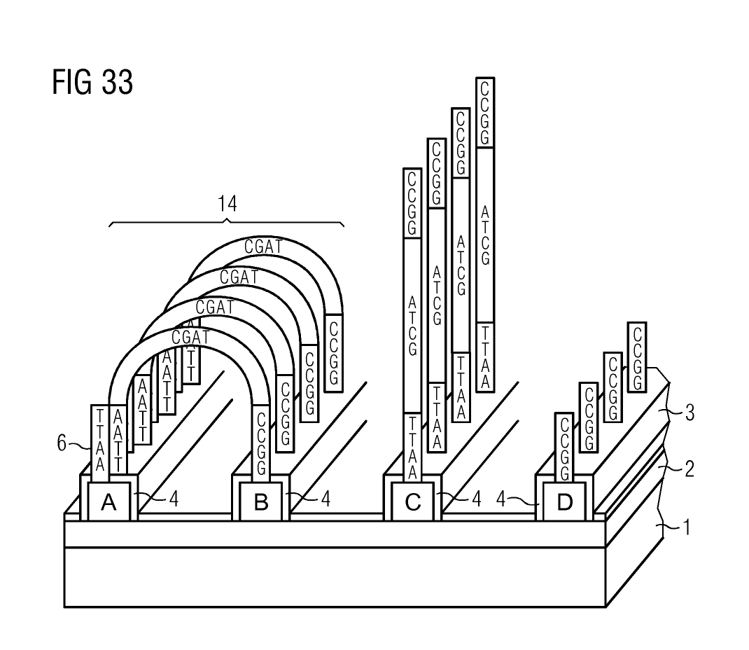

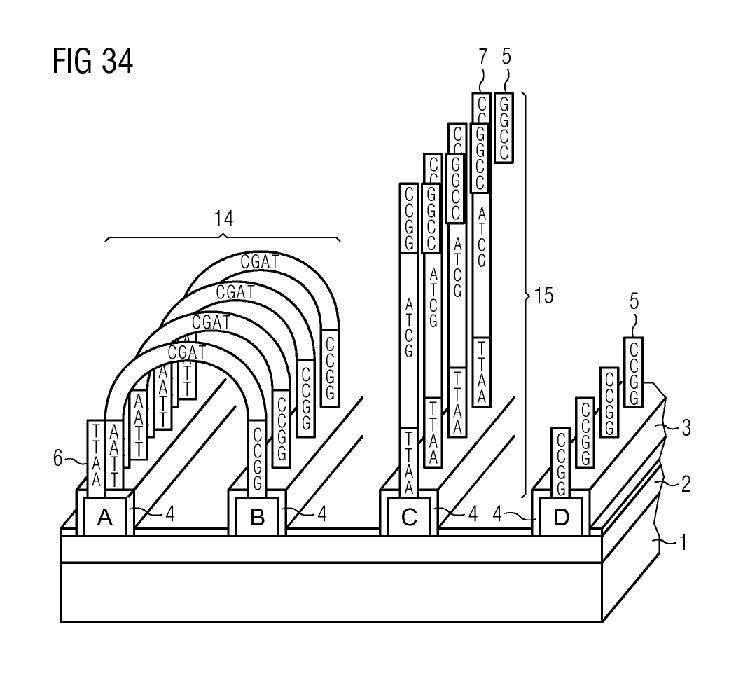

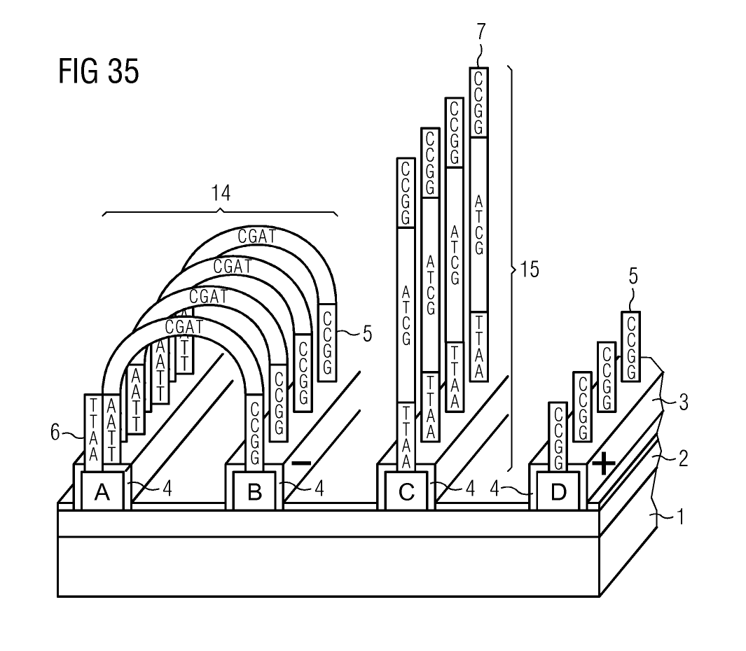

In accordance with the illustration in FIG. 33, the single-stranded DNA molecules 14 bound to the nanowire surface B hybridize to the nucleic acid primers 6 bound to the nanowire surface A by the directed non-stochastic formation of a bridge. The hybridization of the single-stranded DNA molecules 15 covalently bonded to the nanowire surface C to complementary sequence regions 5 of the single-stranded DNA molecules 14 can be prevented by the supply of DNA primer 5, as shown in FIG. 34, or by applying an electric field, as shown in FIG. 35. Possible hybridizations of the sequence regions 7 of the DNA molecules 15 to the sequence regions 5 of the DNA molecules 14 on nanowire B are denatured by the negative pole of the field and by increasing the temperature at points on nanowire B. Furthermore, it is possible to keep the primary binding site 1, i.e. the nucleic acid primer 6, longer than the primary binding site 2, i.e. the nucleic acid primer 5. As a result, the hybridization of the DNA molecules 14 to the nucleic acid primer 6 during a field change or a local temperature increase is stabilized and maintained.

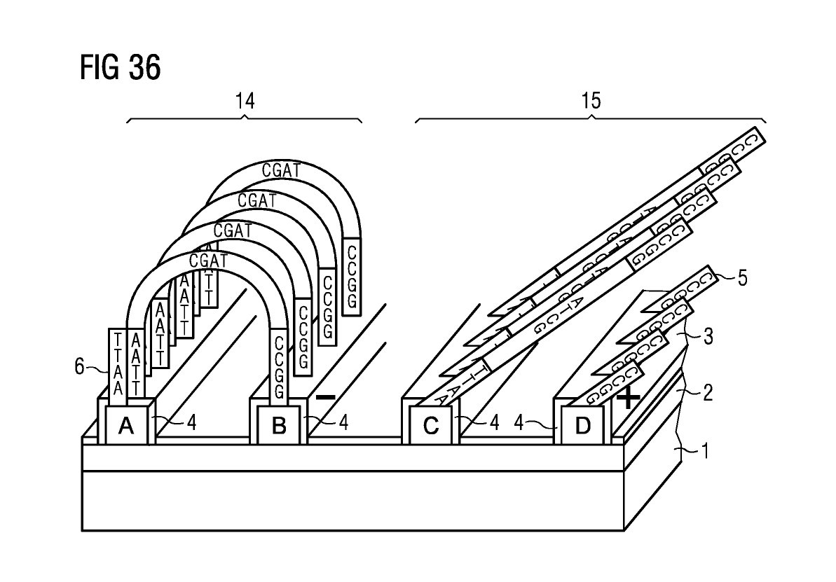

As shown in FIG. 36, a field change takes place, wherein a negative pole is applied to nanowire B and a positive pole is applied to nanowire D. The single-stranded DNA molecules 15 bound to the nanowire surface C and the nucleic acid primers 5 bound to the nanowire surface D are aligned.

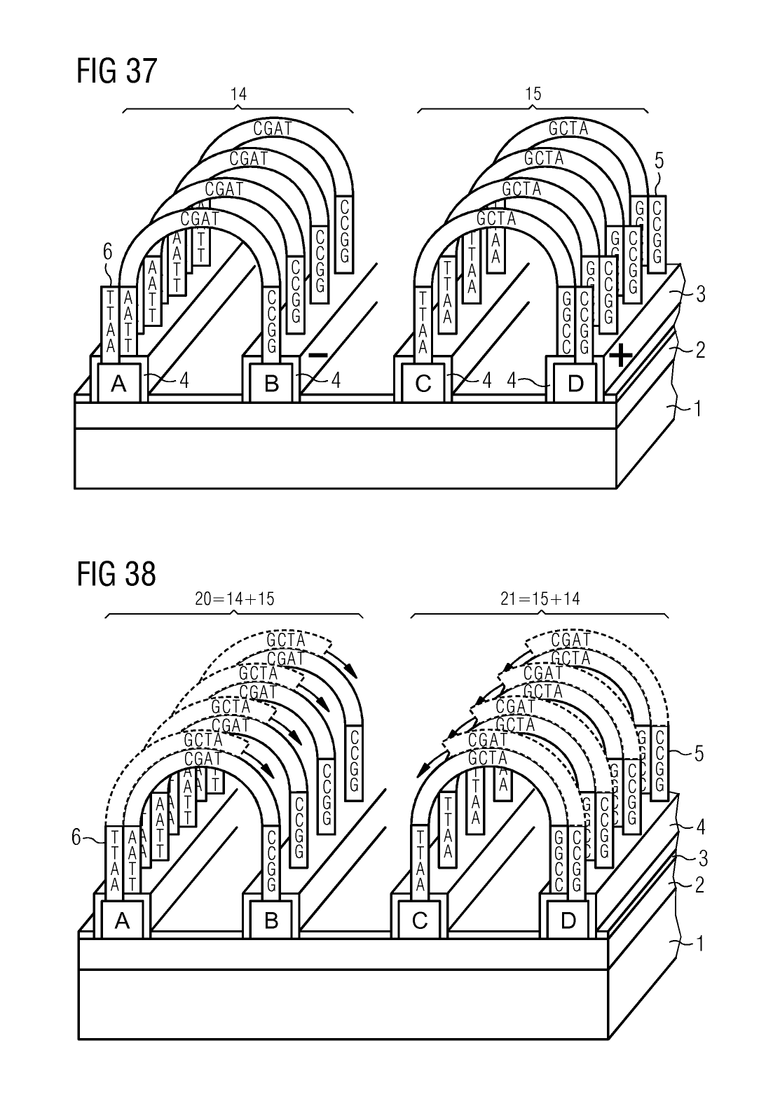

As shown in FIG. 37, the single-stranded DNA molecules 15 hybridize to the nucleic acid primers 5 by the directed, non-stochastic formation of a bridge. This results in the formation of two bridges per nanowire cluster A, B, C, D, in each case comprising forward strand and reverse strand of a DNA 14 and 15.

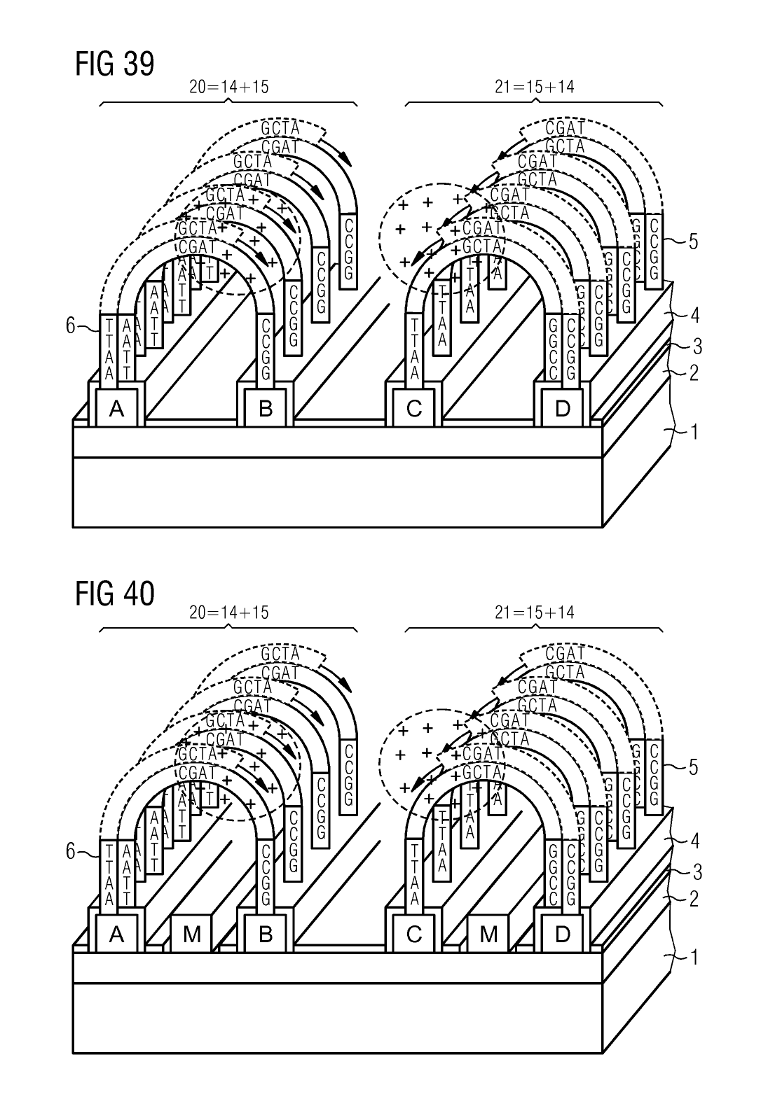

FIG. 38 shows the elongation of the nucleic acid primers 6 and 5. Addition of polymerase and sequential supply of NTPs A, C, T and G result in the elongation of the nucleic acid primer 6 hybridized to the single-stranded DNA molecule 14 with respect to a DNA single strand 15, using the single-stranded DNA module 14 as a template, in order to obtain a DNA double strand 20, and the elongation of the nucleic acid primer 5 hybridized to the single-stranded DNA molecule 15 with respect to a DNA single strand 14, using the single-stranded DNA molecule 15 as a template, in order to obtain a DNA double strand 21, with the counter-strands being constructed step by step. Each time a nucleotide is incorporated, a charge cloud composed of H+ arises, which is represented by (+) signs in FIGS. 39, 40 and 41 and is electrically measured by a nanowire FET. The unipolar nanowire FETs are preferably not operated in inversion, but rather used in the depletion mode. The measurement takes place with a long Debye-Hückel length, usually with a buffer of <1 mmol. The measurement can take place on nanowire A and nanowire B, and respectively on nanowire C and nanowire D. The measurement can also take place in a complementary fashion, p- and n-doped nanowires. The measured signal will be correlated with the supplied nucleotide or the incorporation of a plurality of identical nucleotides will be determined by the signal amplitude.

FIG. 40 shows one embodiment, wherein in this embodiment a further nanowire M is situated below a nucleic acid bridge and between two nanowires A-B or C-D, wherein the two nanowires are connected by a nucleic acid bridge. The further nanowire M is a semiconductor nanowire situated between two metal nanowires, either A-B or C-D. The semiconductor nanowire M is a FET and hence the sensor element. Since the nucleic acid amplification and the nucleic acid sequencing in this embodiment take place on the surfaces of metal nanowires, the semiconductor nanowire M is situated between in each case two such metal nanowires, since the metal nanowires are not part of a FET in this case. For the electronic read-out, therefore, the semiconductor nanowire FET M lies between the metal nanowires.

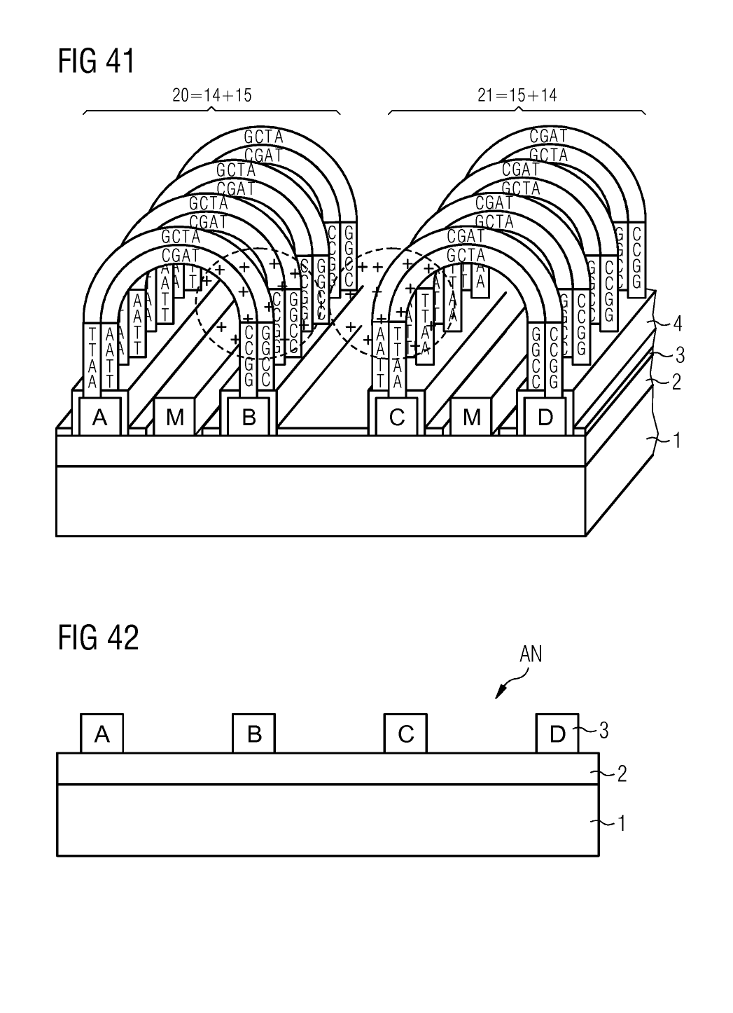

FIG. 41 shows the nucleic acid double strands 20 and 21 after the directed bridge sequencing has ended.

FIG. 42 shows a last work step after the DNA bridge amplification and DNA bridge sequencing. The microfluidic assembly is removed and the chip is regenerated by chemical cleaning. This enables the die to be reused. The costs are thereby reduced.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1. A method for sequencing nucleic acid molecules, comprising:
amplifying one or more nucleic acid molecules; and
after amplification, sequencing the one or more amplified nucleic acid molecules,
wherein amplifying and sequencing are carried out on an array of field effect transistor (FET) sensor elements, comprising an array of nanowires.

2. The method as claimed in claim 1, wherein before amplifying, the nanowires of the array are coated with a polytetrafluoroethylene (PTFE) polymer.

3. The method as claimed in claim 1, wherein before amplifying, the nanowires of the array are coated with a polymer.

4. The method as claimed in claim 3, wherein
the polymer coated on the nanowires is ablated by Joule heating, and
after ablating, a nucleic acid primer is bound to a surface of at least one of the nanowires.

5. The method as claimed in claim 1, wherein
amplifying comprises a directed clonal bridge amplification, and
sequencing comprises a directed bridge sequencing.

6. The method as claimed in claim 5, wherein directed clonal bridge amplification comprises:
hybridizing a single-stranded nucleic acid molecule to a nucleic acid primer, on a surface of at least one of the nanowires.

7. The method as claimed in claim 6, wherein the single-stranded nucleic acid molecule is a forward strand or a reverse strand.

8. The method as claimed in claim 5, wherein
a single-stranded nucleic acid molecule is bound to a surface of a first nanowire,
a nucleic acid primer is bound to a surface of a second nanowire adjacent to the first nanowire, and
directed clonal bridge amplification comprises aligning the single-stranded nucleic acid molecule and the nucleic acid primer, using an electric field.

9. The method as claimed in claim 5, wherein
a single-stranded nucleic acid molecule is bound to a surface of a first nanowire,
a nucleic acid primer is bound to a surface of a second nanowire, the second nanowire being adjacent to the first nanowire, and
directed clonal bridge amplification comprises hybridizing the single-stranded nucleic acid molecule and the nucleic acid primer, by a directed non-stochastic formation of a bridge.

10. The method as claimed in claim 5, wherein
a nucleic acid primer is bound to a surface of first nanowire,
a single-stranded nucleic acid molecule is bound to a surface of a second nanowire, the second nanowire being adjacent to the first nanowire,
the single-stranded nucleic acid molecule is hybridized to the nucleic acid primer, and
directed clonal bridge amplification comprises elongating the nucleic acid primer using the single-stranded nucleic acid molecule as a template, to obtain a nucleic acid double strand.

11. The method as claimed in claim 5, wherein directed clonal bridge amplification comprises:
denaturing a nucleic acid double strand, to obtain first and second single-stranded nucleic acid molecules, the first single-stranded nucleic acid molecule being bound to a surface of a first nanowire, the second single-stranded nucleic acid molecule being bound to a surface of a second nanowire, the second nanowire being adjacent to the first nanowire.

12. The method as claimed in claim 1, wherein amplifying one or more nucleic acid molecules comprises:
(i) selectively binding a first nucleic acid primer to a surface of a first nanowire;
(ii) selectively binding a second nucleic acid primer to a surface of a second nanowire;
(iii) hybridizing a first single-stranded nucleic acid molecule to the first nucleic acid primer;
(iv) elongating the first nucleic acid primer using the first single-stranded nucleic acid molecule as a template, to obtain a first nucleic acid double strand bound to the surface of the first nanowire;
(v) denaturing the first nucleic acid double strand bound to the surface of the first nanowire, to obtain the first single-stranded nucleic acid molecule and to obtain a second single-stranded nucleic acid molecule, the second single-stranded nucleic acid molecule being bound to the surface of the first nanowire, wherein the first single-stranded nucleic acid molecule is removed;
(vi) aligning the second single-stranded nucleic acid molecule bound to the surface of the first nanowire and the second nucleic acid primer bound to the surface of the second nanowire, using an electric field;
(vii) hybridizing the second single-stranded nucleic acid molecule to the second nucleic acid primer by directed non-stochastic formation of a bridge;
(viii) elongating the second nucleic acid primer hybridized to the second single-stranded nucleic acid molecule, using the second single-stranded nucleic acid molecule as a template, to obtain a second nucleic acid double strand;
(ix) denaturing the second nucleic acid double strand to obtain the second single-stranded nucleic acid molecule and to obtain a third single-stranded nucleic acid molecule, the second single-stranded nucleic acid molecule being bound the surface of the first nanowire, the third single-stranded nucleic acid molecule being bound to the surface of the second nanowire; and
(x) cyclically repeating steps (vi)-(ix) in order to amplify nucleic acid molecules of a same sequence on the first and second nanowires.

13. The method as claimed in claim 12, further comprising measuring a H+ charge cloud to determine active FETs for sequencing the nucleic acid molecules.

14. The method as claimed in claim 12, wherein steps (vi)-(ix) are performed in the order listed.

15. The method as claimed in claim 12, wherein step (ii) is performed before step

16. The method as claimed in claim 5, wherein directed bridge sequencing comprises:
aligning single-stranded nucleic acid molecules bound to a surface of a first nanowire and nucleic acid primers bound to a surface of a second nanowire using an electric field, the second nanowire being adjacent to the first nanowire.

17. The method as claimed in claim 5, wherein directed bridge sequencing comprises:
hybridizing single-stranded nucleic acid molecules bound to a surface of a first nanowire to nucleic acid primers bound to a surface of a second nanowire, by directed non-stochastic formation of a bridge, the second nanowire being adjacent to the first nanowire.

18. The method as claimed in claim 5, wherein directed bridge sequencing comprises:
elongating nucleic acid primers bound to one nanowire surface and hybridized to single-stranded nucleic acid molecules bound to another, adjacent nanowire surface, using the single-stranded nucleic acid molecules as a template, in order to obtain nucleic acid double strands.

19. The method as claimed in claim 1, wherein sequencing the one or more amplified nucleic acid molecules comprises a directed bridge sequencing of uniform DNA clusters using a pulsed single nucleotide supply and electrical signal read-out.

20. The method as claimed in claim 1, wherein sequencing the one or more amplified nucleic acid molecules comprises:
selectively binding second nucleic acid primers to a surface of a first nanowire;
selectively binding first nucleic acid primers to a surface of a fourth nanowire;
aligning first single-stranded nucleic acid molecules bound to a surface of a second nanowire and the second nucleic acid primers, using an electric field, the second nanowire being adjacent to the first nanowire;
hybridizing the first single-stranded nucleic acid molecules to the second nucleic acid primers, by a directed non-stochastic formation of a bridge;
aligning second single-stranded nucleic acid molecules bound to a surface of a third nanowire and the first nucleic acid primers, using a field change of the electric field, the third nanowire being adjacent to the fourth nanowire;
hybridizing the second single-stranded nucleic acid molecules to the first nucleic acid primers, by a directed non-stochastic formation of a bridge;
elongating the second nucleic acid primers hybridized to the first single-stranded nucleic acid molecules, using the first single-stranded nucleic acid molecules as a template, to obtain first nucleic acid double strands, and elongating the first nucleic acid primers hybridized to the second single-stranded nucleic acid molecules, using the second single-stranded nucleic acid molecules as a template, to obtain second nucleic acid double strands,
wherein elongating the second and first nucleic acid primers comprises incorporating sequentially at least one nucleoside triphosphate at a 3′ end of each of the second and first nucleic acid primers; and
detecting incorporation of nucleoside triphosphates by measuring a H+ charge cloud in a vicinity of the nucleic acid bridges.

21. The method as claimed in claim 20, wherein at least one measuring nanowire is provided below the nucleic acid bridges, the at least one measuring nanowire being used to measure incorporation of nucleoside triphosphates.

22. The method as claimed in claim 21, wherein
a first measuring nanowire is situated between the first and second nanowires,
a second measuring nanowire is situated between the third and fourth nanowires, and
the first and second measuring nanowires are nanowire FETs having no polymer coating.

23. A sequencing device comprising an array of field effect transistor (FET) sensor elements, the array being formed form an array of nanowires, wherein
wherein each FET comprises at least one nanowire, or
wherein each FET is positioned between two nanowires, or
wherein one nanowire of each nanowire pair is a nanowire FET, and
wherein one or more nucleic acids are bound to a surface of at least one nanowire for amplifying and sequencing the one or more nucleic acid molecules.

24. The sequencing device as array as claimed in claim 23, wherein the device is a chip comprising one or more arrays of field effect transistor (FET) sensor elements.

25. The sequencing device as claimed in claim 24, further comprising a microfluidic system.

26. The sequencing device as claimed in claim 23, wherein
the device is a kit, and
the device further comprises amplifying reagents or sequencing reagents.
